SEQUENCE LISTING (18 sequences)

SEQ ID NO: 1 - RNA, 20 nt, synthetic construct
  source 1..20: mol_type = other RNA
ggggccactagggacaggat

SEQ ID NO: 2 - DNA, 27 bp, synthetic construct
  source 1..27: mol_type = other DNA
ttcccaggcaggtcctgctttctctga

SEQ ID NO: 3 - DNA, 20 bp, synthetic construct
  source 1..20: mol_type = other DNA
gtgggcttgtactcggtcat

SEQ ID NO: 4 - DNA, 23 bp, synthetic construct
  source 1..23: mol_type = other DNA
tctaacaggtaccatgtggggtt

SEQ ID NO: 5 - DNA, 21 bp, synthetic construct
  source 1..21: mol_type = other DNA
atctaggtagccacaggaggc

SEQ ID NO: 6 - DNA, 33 bp, synthetic construct
  source 1..33: mol_type = other DNA
atataagcttatggtgagcaagggcgaggagct

SEQ ID NO: 7 - DNA, 29 bp, synthetic construct
  source 1..29: mol_type = other DNA
atgccttgtacagctcgtccatgccgaga

SEQ ID NO: 8 - RNA, 20 nt, synthetic construct
  source 1..20: mol_type = other RNA
agttaatctgttctaggcgg

SEQ ID NO: 9 - RNA, 20 nt, synthetic construct
  source 1..20: mol_type = other RNA
cgggggtcatctggataact

SEQ ID NO: 10 - DNA, 21 bp, synthetic construct
  source 1..21: mol_type = other DNA
ggtaccttctcgctcatcatc

SEQ ID NO: 11 - DNA, 19 bp, synthetic construct
  source 1..19: mol_type = other DNA
agtggtcattgcgcttctt

SEQ ID NO: 12 - RNA, 19 nt, synthetic construct
  source 1..19: mol_type = other RNA
gatggagtctcatggcaca

SEQ ID NO: 13 - RNA, 19 nt, synthetic construct
  source 1..19: mol_type = other RNA
tggatgacttgcttgggta

SEQ ID NO: 14 - RNA, 19 nt, synthetic construct
  source 1..19: mol_type = other RNA
aggtatggagagtcggctt

SEQ ID NO: 15 - RNA, 19 nt, synthetic construct
  source 1..19: mol_type = other RNA
aggcagacctgttatccta

SEQ ID NO: 16 - DNA, 757 bp, Homo sapiens
  source 1..757: mol_type = genomic DNA
ctcgctccgccaccacaggggggcgacacggcgcagcgccgaaagagttaatctgttctaggcgggggaagtgcgggcttgggggtgggaggcaggacgcttagcttggcctggagctgcgccccgcgctggacgctcggattccgctcgctgcctggactcagagcacaattgcgtttcctgcgggttatttttggcgtgggaacgcggggagtacggcggtgagaaaggctgaagctgccagcgccgctgacgggccccttcctgtattttacacctttcgcgaattccgctcctttggaaagggaataatggctttgggatgttgttctgacacagaggaaaaggatatttcagcagcacaacaattctcactttgaaaaggaaaaaagaaaaccattacccacctctggaggcagaacccctgaatgggcaccaaaggaccccctgctcccagggtcctctctagcctggggagcttttctttctttttctcttttttccattttgacctcttttcctctttcccctccctatctgcctccaagaccctgggatatcttaacatccttctattgtcccctttttgaatactatcaggccccctgcacatgcacacacgtagggcagctacgtagcggggctttgggtccctctggcctgttcttgctggcaggcgggggtcatctggataactgggctgattggttggctgatcaccatcatcacagccaagaaggacattggccagccgtca

SEQ ID NO: 17 - DNA, 632 bp, Homo sapiens
  source 1..632: mol_type = genomic DNA
cgggggaagtgcgggcttgggggtgggaggcaggacgcttagcttggcctggagctgcgccccgcgctggacgctcggattccgctcgctgcctggactcagagcacaattgcgtttcctgcgggttatttttggcgtgggaacgcggggagtacggcggtgagaaaggctgaagctgccagcgccgctgacgggccccttcctgtattttacacctttcgcgaattccgctcctttggaaagggaataatggctttgggatgttgttctgacacagaggaaaaggatatttcagcagcacaacaattctcactttgaaaaggaaaaaagaaaaccattacccacctctggaggcagaacccctgaatgggcaccaaaggaccccctgctcccagggtcctctctagcctggggagcttttctttctttttctcttttttccattttgacctcttttcctctttcccctccctatctgcctccaagaccctgggatatcttaacatccttctattgtcccctttttgaatactatcaggccccctgcacatgcacacacgtagggcagctacgtagcggggctttgggtccctctggcctgttcttgctggcaggcgggggtcatctggata

SEQ ID NO: 18 - RNA, 19 nt, synthetic construct
  source 1..19: mol_type = other RNA
tggtttacatgtcgactaa

(19) United States
(12) Patent Application Publication
(10) Pub. No.: US 2024/0191189 A1
(43) Pub. Date: Jun. 13, 2024

(54) REAGENTS AND METHODS FOR PRODUCING ARTERIAL HEMOGENIC ENDOTHELIUM, HEMATOPOIETIC PROGENITORS, AND LYMPHOID CELLS THEREBY

(71) Applicant: Wisconsin Alumni Research Foundation, Madison, WI (US)

(72) Inventors: Igor Slukvin, Verona, WI (US); Akhilesh Kumar, Middleton, WI (US); Saritha Sandra D'Souza, Middleton, WI (US)

(21) Appl. No.: 18/537,479

(22) Filed: Dec. 12, 2023

Related U.S. Application Data
(60) Provisional application No. 63/387,052, filed on Dec. 12, 2022.
(60) Provisional application No. 63/520,241, filed on Aug. 17, 2023.

(51) Int. Cl. C12N 5/0783; C12N 5/0781

(52) U.S. Cl. CPC C12N 5/0636; C12N 5/0635; C12N 2506/45; C12N 2510/00; C12N 2513/00

(57) ABSTRACT

The present disclosure provides reagents and methods for producing arterial hemogenic endothelium, hematopoietic progenitor, and lymphoid cell cultures. Pharmaceutical compositions comprising arterial hemogenic endothelium cells, hematopoietic progenitor cells, and lymphoid cells produced by the reagents and methods of the invention and therapeutic methods using these pharmaceutical compositions are also provided.

### RELATED APPLICATIONS

This application claims priority to U.S. provisional application No. 63/387,052, filed Dec. 12, 2022 and U.S. provisional application No. 63/520,241, filed Aug. 17, 2023, the disclosures of each of which are expressly incorporated by reference herein in its entirety.

### STATEMENT REGARDING FEDERALLY SPONSORED RESEARCH

This invention was made with government support under HL 142665 awarded by the National Institute of Health. The government has certain rights in the invention.

### INCORPORATION BY REFERENCE OF SEQUENCE LISTING PROVIDED ELECTRONICALLY

This application contains a Sequence Listing submitted as an electronic text file named “22-2159-US-PRO2_SequenceListing.xml,” having a size in bytes of 17,971 bytes, and created on Aug. 16, 2023. The information contained in this electronic file is hereby incorporated by reference in its entirety.

### BACKGROUND OF THE DISCLOSURE

Human embryonic stem cells (hESCs) and induced pluripotent stem cells (iPSCs) represent a robust source of lymphoid and myeloid cells for immunotherapies, which can be modified with the help of genetic engineering technologies to meet specific clinical need (Clapes and Robin, 2012, Regen Med. 7: 349-368). Multiple studies have been reported on generating hematopoietic progenitors (HPs) with a hematopoietic stem cell (HSC) phenotype and limited engraftment potential from human pluripotent stem cells (hPSCs; Ledran et al., 2008, Cell Stem Cell 3: 85-98; Sugimura et al., 2017, Nature 545: 432-438; Wang et al., 2005, J. Exp. Med. 201: 1603-1614). However, immune reconstitution and consistent engraftment with recapitulation of the full spectrum of terminally differentiated hematopoietic cells have been unsuccessful.

Hematopoietic development in the vertebrate embryo occurs in multiple waves from hemogenic endothelium (HE) intermediates in several extra- and intra-embryonic locations including yolk sac vessels, the aorta, vitelline and umbilical arteries, placenta, endocardium, and possibly head and somatic vasculature. Although the earliest waves of hematopoiesis, primitive and erythromyeloid, are NOTCH-independent and occur in different vessel types, initiation of HSC and lymphoid cell formation requires NOTCH signaling and only occurs in arteries (Bertrand et al., 2010, Nature 464: 108-111; de Bruijn et al., 2000, EMBO J 19: 2465-2474; Gordon-Keylock et al., 2013, Blood 122: 238-2345; Yzaguirre and Speck, 2016, Devel. Dyn 245: 1011-1028). During vascular development, arterial fate is specified following induction of delta-like 4 (DLL4) expression by VEGF (Lawson et al., 2001, Development 128: 3675-3683), wherein DLL4 expression is initiated by signaling through a highly conserved VEGF-responsive artery-specific enhancer located within the third intron of DLL4 (eDLL4i3) which is controlled by ETS, SOXF and RBPJ factors (Sacilotto et al., 2013, Proc. Natl. Acad. Sci. USA 110: 11893-11898; Wythe et al., 2013, Dev. Cell 26: 45-58).

There are several hematopoietic differentiation systems that have been reported to generate arterial hemogenic endothelium and hematopoietic progenitors, including co-culture with feeder cells (Choi et al., 2009, J. Clin. Invest. 119: 2818-282), embryoid bodies (EB; Daley, 2003, Ann. N.Y. Acad. Sci. 996: 122-131; Lu et al., 2007, Cell Biol. Int. 29:817-825), two-dimensional culture (Uenishi et al., 2014, Nat. Commun. 9: 1828; Feng et al., 2014, Stem Cell Reports 3: 817-831), and forced expression of transcription factor (Sugimura et al., 2017, Nature 545: 432-438). However due to the undefined nature of feeder cells, large variability of EB, and requirements of large surface areas (2D systems), scale up opportunities are limited and these methods have been unsuitable for therapeutic manufacture applications.

There is a need in this art to further develop reagents, including cell culture media and conditions, and methods for reliably effecting hematopoietic differentiation and hematopoietic cells resulting therefrom that can be produced on a useful scale for producing hematopoietic cells for therapeutic intervention and other purposes.

### BRIEF SUMMARY OF THE DISCLOSURE

Provided herein are reagents and methods for reliably effecting hematopoietic differentiation and hematopoietic cells resulting therefrom that can be produced on a useful scale for providing hematopoietic cells for therapeutic intervention.

In particular embodiments, provided herein are methods for producing a spheroid containing pluripotent stem cells for hematopoietic progenitor cell differentiation, comprising the steps of:
a) dispersing pluripotent stem cells (PSCs) into a single cell suspension of spheroid medium, wherein the medium contains 25% Dulbecco's Modified Eagle medium, 25% Ham's F-12 medium, 10% BIT 9500 supplements, 2 mM L-alanyl-L-glutamine dipeptide, 0.1 mM Non-essential Amino Acids (NEAA), 100 μM monothioglycerol (MTG), 100 μg/ml ascorbic acid, and 40% ES-Cult M3120 comprising 2.6% methylcellulose in Iscove's Modified Dulbecco's Medium,
b) apportioning the cell suspension into individual, separated droplets each comprising about 2,500-3,500 cells in 30-40 μl spheroid medium onto a solid surface in a sealable vessel, wherein the vessel is thereafter sealed and inverted wherein the droplets are hanging from the solid surface; and
c) incubating the droplet-comprising vessel for 20-24 hours in an incubator at cell-sustaining temperature and humidity conditions to produce cell-comprising spheroids.

Also provided herein are methods of producing hematopoietic progenitor cells from the spheroid containing pluripotent stem cells generated as described above, further comprising the steps of:
a) harvesting cell-comprising spheroids with a buffer medium that is phosphate-buffered saline or iPSC culture medium by centrifugating and resuspending the spheroids in IF9S medium supplemented with bone morphogenetic protein 4 (BMP4), vascular endothelial growth f factor (VEGF), fibroblast growth factor (FGF), Activin A and Rho Kinase (ROCK) inhibitor, and thereafter plating 140-160 of the spheroids on coated culture plates per 50-60 cm² surface area of the plates;
b) culturing the plated spheroids/cells under hypoxic conditions;
c) after 46-50 hours of hypoxic culture, changing the culture medium to IF9S supplemented with VEGF, FGF, BMP4, glycogen synthase kinase 3 inhibitor CHIR99021, and activin receptor-like kinase receptors inhibitor SB-431542, and again incubating the spheroids/cells under hypoxic conditions;
d) after 24 hours of hypoxic culture, changing the culture medium to IF9S medium supplemented with VEGF, FGF, and BMP4 and again incubating the spheroids/cells under hypoxic conditions;
e) after 48 hours of hypoxic culture, changing the culture medium to IF9S medium supplemented with stem cell factor (SCF), VEGF, thrombopoietin (TPO), interleukin 6 (IL6), FGF, and interleukin 3 (IL3), and incubating the spheroids/cells under normoxic conditions;
f) after 48 hours of normoxic culture, adding IF9S medium supplemented with SCF, VEGF, TPO, IL6, FGF, and IL3 to the culture medium and incubating the spheroids/cells under normoxic conditions to produce hematopoietic progenitors; and
g) two to four days thereafter, harvesting floating hematopoietic progenitor cells from the culture medium or dislodging hematopoietic progenitor cells attached to the surface or both, and separating hematopoietic progenitors by biomarker-based cell sorting, wherein the cells are differentiated into hemogenic endothelium cells 48 hours after step (d) and at least 50% of the hemogenic endothelium cells are arterial hemogenic endothelium cells (AHEs).

In some embodiments of this method, the plates are coated with collagen, DLL4-Fc fusion proteins, or Jagged canonical Notch ligand 1 (JAG1)-Fc fusion proteins. In some embodiments, VEGF is present in the culture medium at concentrations of between 25-50 ng/ml, Activin A is present in the culture medium at concentrations of between 10-15 ng/ml, ROCK inhibitor is present in the culture medium at concentrations of between 1-10 μM, CHIR99021 is present in the culture medium at concentrations of between 1-3 μM, SB-431542 is present in the culture medium at concentrations of between 3-5 μM, SCF is present in the culture medium at concentrations of between 25-50 ng/ml, TPO is present in the culture medium at concentrations of between 25-50 ng/ml, IL6 is present in the culture medium at concentrations of between 25-50 ng/ml, and IL3 is present in the culture medium at concentrations of between 5-10 ng/ml. In step (a), BMP4 is present in the culture medium at concentrations of between 10-50 ng/ml and fibroblast growth factor (FGF) is present in the culture medium at concentrations of between 10-50 ng/ml. In step (c) and step (d), BMP4 is present in the culture medium at concentrations of between 10-20 ng/mL and fibroblast growth factor (FGF) is present in the culture medium at concentrations of between 10-50 ng/ml. In step (e) and step (f), fibroblast growth factor (FGF) is present in the culture medium at concentrations of between 10-20 ng/ml.

In preferred embodiments, VEGF is present in the culture medium at a concentration of 50 ng/mL, Activin A is present in the culture medium at a concentration of 15 ng/ml, ROCK inhibitor is present in the culture medium at a concentration of 1 μM, CHIR99021 is present in the culture medium at a concentration of 1 μM, SB-431542 is present in the culture medium at a concentration of 3 μM, SCF is present in the culture medium at a concentration of 50 ng/ml, TPO is present in the culture medium at a concentration of 50 ng/ml, IL6 is present in the culture medium at a concentration of 50 ng/ml, and IL3 is present in the culture medium at a concentration of 10 ng/ml. In step (a), BMP4 is present in the culture medium at a concentration of 50 ng/ml, and fibroblast growth factor (FGF) is present in the culture medium at a concentration of 50 ng/ml. In step (c) and step (d), BMP4 is present in the culture medium at a concentration of 10 ng/mL, and fibroblast growth factor (FGF) is present in the culture medium at a concentration of 50 ng/ml. In step (e) and step (f), fibroblast growth factor (FGF) is present in the culture medium at a concentration of 10 ng/ml.

Also provided herein is a hematopoietic progenitor cell culture produced by the methods described above, in which DLL4 expression and activity is reduced in the PSCs, and compositions comprising such cells.

In other aspects provided herein are pharmaceutical compositions comprising hematopoietic progenitor cells, particularly as produced by the disclosed methods, the compositions further comprising pharmaceutically or therapeutically acceptable excipients and adjuvants.

Provided herein is a method of producing T cells from hematopoietic progenitor cells derived from the spheroid containing pluripotent stem cells, further comprising:
a) culturing the hematopoietic progenitor cells with feeder cells expressing Notch-ligand delta like 4 (DLL4) in minimum essential Eagle medium with alpha modification (MEMα) supplemented 20% fetal bovine serum, SCF, FMS-like tyrosine kinase 3 ligand (FLT-3L), and interleukin 7 (IL7) for three weeks; and
b) harvesting floating cells and passaging them weekly onto the feeder cells expressing DLL4 to produce T cells.

In some embodiments of the method of producing T cells from hematopoietic progenitor cells derived from the spheroid containing pluripotent stem cells described above, SCF is present in the culture medium at concentrations between 10-25 ng/ml, FLT-3L is present in the culture medium at concentrations between 5-10 ng/ml, and IL7 is present in the culture medium at concentrations between 5-10 ng/ml. In preferred embodiments, SCF is present in the culture medium at a concentration of 10 ng/ml, FLT-3L is present in the culture medium at a concentration of 5 ng/ml, and IL7 is present in the culture medium at a concentration of 5 ng/ml.

Provided herein is a method of increasing T cell production from the hematopoietic progenitor cells derived from the spheroid containing pluripotent stem cells described above, and further comprising reducing DLL4 expression or activity in the PSCs and reducing GAS5 expression in the HEs.

Provided herein is a method of increasing T cell production from the hematopoietic progenitor cells (HPs) derived from the spheroid containing pluripotent stem cells described above, and further comprising reducing DLL4 expression or activity in the PSCs, wherein the plates are coated with JAG-Fc fusion proteins. In some embodiments, the JAG1-Fc fusion proteins are used at concentrations between 0.5-1.5 μg per cm² of the plate's surface area.

Provided herein is a method of producing B cells from hematopoietic progenitor cells derived from the spheroid containing pluripotent stem cells, further comprising:
a) culturing the hematopoietic progenitor cells with feeder cells in minimum essential Eagle medium with alpha modification (MEMα) supplemented 20% fetal bovine serum, FMS-like tyrosine kinase 3 ligand (FLT-3L), and interleukin 7 (IL7) for three weeks; and
b) harvesting floating cells and passaging them weekly onto the feeder cells to produce B cells.

In some embodiments of the method of producing B cells comprising the method of producing hematopoietic progenitor cells from the spheroid containing pluripotent stem cells described above, FLT-3L is present in the culture medium at concentrations between 5-10 ng/ml, and IL7 is present in the culture medium at concentrations between 5-10 ng/ml. In preferred embodiments, FLT-3L is present in the culture medium at a concentration of 5 ng/ml, and IL7 is present in the culture medium at a concentration of 5 ng/ml.

Provided herein is a method of increasing B cell production from the hematopoietic progenitor cells derived from the spheroid containing pluripotent stem cells described above, and further comprising reducing DLL4 expression or activity in the PSCs.

Provided herein is a method of increasing B cell production from the hematopoietic progenitor cells (HPs) derived from the spheroid containing pluripotent stem cells described above, and further comprising reducing DLL4 expression or activity in the PSCs, wherein the plates are coated with JAG-Fc fusion proteins. In some embodiments, the JAG1-Fc fusion proteins are used at a concentration between 0.5-1.5 μg per cm² of the plate's surface area.

Provided herein is a method of producing T cells from hematopoietic progenitor cells derived from the spheroid containing pluripotent stem cells, and further comprising reducing DLL4 expression or activity in the PSCs and reducing GAS5 expression in the hemogenic endothelium cells (HEs), and differentiating the hematopoietic progenitor cells into the T cells thereby.

Provided herein is a method of producing T cells from hematopoietic progenitor cells derived from the spheroid containing pluripotent stem cells, and further comprising reducing DLL4 expression or activity in the PSCs and differentiating the hematopoietic progenitor cells into the T cells thereby, wherein the plates are coated with JAG-Fc fusion proteins. In some embodiments, JAG1-Fc fusion proteins are used at a concentration between 0.5-1.5 μg per cm² of the plate's surface area.

Provided herein is a method of producing B cells from hematopoietic progenitor cells from the spheroid containing pluripotent stem cells, and further comprising reducing DLL4 expression or activity in the PSCs and differentiating the hematopoietic progenitor cells into the B cells thereby, wherein the plates are coated with JAG-Fc fusion proteins. In some embodiments, JAG1-Fc fusion proteins are used at a concentration between 0.5-1.5 μg per cm² of the plate's surface area.

Provided herein is a method of producing T cells from pluripotent stem cells (PSCs) comprising reducing DLL4 expression or activity in the PSCs and reducing GAS5 expression in hemogenic endothelium cells (HEs) derived from the PSCs, and differentiating the HEs into the T cells thereby.

Provided herein is a method of producing T cells from pluripotent stem cells (PSCs) comprising reducing DLL4 expression or activity in the PSCs and differentiating HPs derived from PSCs into the T cells thereby, wherein the PSCS are cultured on JAG-Fc fusion proteins coated plates until the HPs are obtained. In some embodiments, the JAG1-Fc fusion proteins are used at a concentration between 0.5-1.5 μg per cm² of the plate's surface area.

Provided herein is a method of producing B cells from pluripotent stem cells (PSCs) comprising reducing DLL4 expression or activity in the PSCs, and differentiating the PSCs into the B cells thereby.

Provided herein is a method of producing B cells from pluripotent stem cells (PSCs) comprising reducing DLL4 expression or activity in the PSCs and differentiating hematopoietic progenitor cells (HPs) derived from PSCs into the B cells thereby, wherein the PSCs are cultured on JAG1-Fc fusion proteins coated plates until the HPs are obtained. In some embodiments, JAG1-Fc fusion proteins are used at a concentration between 0.5-1.5 μg per cm² of the plate's surface area.

In certain embodiments, DLL4 expression is reduced in the PSCs in any of these methods by deletion of an artery-specific enhancer within intron 3 of DLL4 (eDLL4i3). In certain embodiments, DLL4 expression is reduced in the PSCs in any of these methods by using CRISPR Cas9, transcription activator-like effector nuclease (TALEN), zinc finger nuclease (ZNF), short hairpin RNA (shRNA), or short interference RNA (siRNA). In certain embodiments, DLL4 activity is reduced by in the PSCs in any of these methods by having antibodies competitively bind to Notch receptor expressed by pluripotent stem cells. In certain embodiments, the GAS5 expression is reduced in the hemogenic endothelium cells in any of these methods by using CRISPR Cas9, TALEN, ZNF, shRNA, or siRNA.

In other aspects, provided herein is a pharmaceutical composition comprising the T cells or B cells produced by any of these methods, and pharmaceutically or therapeutically acceptable excipients and adjuvants.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

The disclosure will be better understood and features, aspects, and advantages other than those set forth above will become apparent when consideration is given to the following detailed description thereof. Such detailed description refers to the following drawings.

FIG. 1 is a diagram of hematopoietic differentiation using the spheroid hanging drop method (HDM) disclosed herein.

FIG. 2A are Assay for Transposase-Accessible Chromatin (ATAC) and H3K4me3 (tri-methylation at the 4th lysine residue of the histone H3 protein epigenetic modification) signals that show that eDLL413 was active in hemogenic endothelium (HE) and responded to SOX17 overexpression.

FIG. 2B shows the construct used to generate the eDLL4i3 reporter line (eDLL4i3:VENUS reporter). The reporter was used to present a real-time arterial hemogenic endothelium (AHE)-specific map of hematopoietic differentiation.

FIG. 2C are eDLL413:Venus reporter cell lines validated by Southern blot. The asterisk (*) indicates a correct band size for properly edited clone. The arrow (→) indicates a proper size of DNA fragment of unedited wild type hPSC.

FIG. 2D is a diagram of a three-dimensional (3D)-spheroid platform for hematopoietic differentiation in defined conditions as disclosed herein, wherein D was the day of differentiation.

FIG. 2E is a phase-contrast image of 3D-spheroids formed by the hanging drop method as disclosed herein and post-transferred spheroid in tissue culture plates/dishes.

FIG. 2F shows fluorescent images of the eDLL4i3 reporter line showing expression of Venus during different stages of differentiation.

FIG. 2G shows flow cytometric analysis of cells having the D4 immature HE phenotype.

FIG. 2H shows the percentage of Venus cells in different D4 HE subsets.

FIG. 2I shows flow cytometric analysis of expression of arterial cell markers and a reporter construct (eDLL4i3:VENUS) on Day 5 of differentiation.

FIG. 2J are bar graphs showing the percentage of VENUS marker in different subsets of HE on Day 5 of differentiation. Results are expressed as mean±SD, n=3 experiments, **p<0.001).

FIG. 2K is a graph showing colony-forming cell (CFC) potential of hematopoietic progenitor (HP) cells collected following Day 5 of culture for VENUS expressing (VENUS) and non-expressing (VENUS″) cells and further culturing on OP9 or OP9-DLL4 stroma for five days. M: macrophage colony forming cells. GM: granulocyte and macrophage colony forming cells. GEMM: granulocyte, erythroid, megakaryocyte, and macrophage colony forming cells. G: granulocyte colony forming cells. E: erythroid colony forming cells.

FIG. 2L illustrates results of DLL4 and VENUS expression using single cell RNAseq. eDLL413-enhancer activation correlates with eDLL413:VENUS expression, confirming specificity of this reporter.

FIG. 2M shows results of flow cytometric analysis of T cell marker gated on CD45+ cells.

FIG. 2N are bar graphs showing T cell and natural killer (NK) cell potential for Day 5 subsets as assessed for T cell and NK markers by flow cytometry gated on CD45+ cells. The graphs show the total number of sorted cells from a total population of 10,000 cells. Note that CD144 is pan-endothelial marker, CD43 is pan-hematopoietic marker, and CD73 is used to separate CD73−hemogenic endothelium from CD73+ non-hemogenic endothelium.

FIG. 3A shows a diagram of a 3D-spheroid as provided herein for differentiation Day 4 (D4) under specified conditions.

FIG. 3B shows the results of flow cytometry of D4 immature HE cells.

FIG. 3C are bar graphs showing percentages and total number of VE-cadherin or cadherin-5 (pan-endothelial cell marker) expressing (VEC+) cells and arterial hemogenic endothelium (AHE) cells on D4 of differentiation from 10,000 hESCs, where values of each are displayed as mean±SD, n=3. **p<0.01.

FIG. 3D show results of flow cytometric analysis of HPs collected following culture of D4 HE on OP9 or OP9-DLL4 for five days.

FIG. 3E are bar graphs showing percentages of CD45 and CD43 cells in HP after co-culture of D4 HE on OP9 or OP9-DLL4 for five days, wherein the values are displayed as mean±SD, n=3. *p>0.05, **p<0.01.

FIG. 3F are pie charts showing CFC potential of HP collected following culture of day 4 HE cultured on OP9 or OP9-DLL4 for five days. M: macrophage colony forming cells. GM: granulocyte and macrophage colony forming cells. GEMM: granulocyte, erythroid, megakaryocyte, and macrophage colony forming cells. G: granulocyte colony forming cells. E: erythroid colony forming cells.

FIG. 3G is a bar graph showing numbers of CFC arising from 10,000 HP cells following culture of day 4 HE cultured on OP9 or OP9-DLL4 for five days.

FIG. 3H show results of flow cytometric analyses of T cell markers on T cells from day 4 HE.

FIGS. 3I and 3J are bar graphs showing total number of T cells generated from day 4 HE cells, wherein the values are displayed as mean±SD, n=3, ***p<0.001.

FIG. 3K show the results of flow cytometric analysis of NK markers on NK cells from day 4 HE.

FIGS. 3L and 3M are bar graphs showing the total number of NK cells generated from day 4 HE cells, where the values are displayed as mean±SD, n=3.

FIG. 4A is a diagram illustrating the experimental design as set forth in the Examples regarding day 5 and day 8 hematopoietic differentiation.

FIG. 4B shows phage contrast photomicrographs of arterial hemogenic endothelium (AHE) cells and the results of flow cytometric analysis of day 5 mature AHE.

FIG. 4C a graphical representation of the effect of eDLL413 knockout on the percentage and total numbers of VEC+ cells and AHE cells on day 5 of differentiation from 10,000 hESCs, wherein the values show a decrease and are displayed as mean±SD, n=3, *p<0.05, **p<0.01, and ***p<0.001.

FIG. 4D shows bar graphs illustrating that eDLL413 knockout also decreases the percentage and total numbers of AHE subsets cells (DLL4⁺CXCR4⁺, DLL4⁺CXCR4⁻, and DLL4⁻CXCR4⁻) on D5 of differentiation from 10,000 hESCs, wherein the values are displayed as mean±SD, n=3, *p<0.05, **p<0.01, and ***p<0.001.

FIG. 4E are pie charts showing CFC potential of HP collected following culture of D5 AHE on OP9 or OP9-DLL4 for five days.

FIG. 4F is a bar graph showing numbers of CFC arising from 10,000 HP cells following culture of D5 AHE cultured on OP9 or OP9-DLL4 for five days.

FIG. 4G shows flow cytometric results demonstrating T cell markers on T cells from day 5 AHE.

FIG. 4H are bar graphs showing total number of T cells generated from day 5 AHE, wherein the values are displayed as mean±SD, n=3, *p<0.05 and ***p<0.001.

FIG. 4I shows phage contrast photomicrographs of round floating cells and the results of flow cytometric analysis showing expression of hematopoietic markers on day 8 of differentiation.

FIG. 4J are bar graphs showing percentage and total number of hematopoietic progenitor cells and subsets thereof on day 8 of differentiation, wherein the values are displayed as mean±SD, n=3, *p<0.05, **p<0.01, and ***p<0.001.

FIG. 4K is a bar graph and FIG. 4L is a pie chart showing CFC potential of total floating HP cells collected on day 8 of differentiation. M: macrophage colony forming cells. GM: granulocyte and macrophage colony forming cells. GEMM: granulocyte, erythroid, megakaryocyte, and macrophage colony forming cells. G: granulocyte colony forming cells. E: erythroid colony forming cells.

FIG. 4M shows the results of flow cytometric analysis of T cell markers in T cell differentiation cultures from total floating day 8 HPs.

FIG. 4N are bar graphs showing total number of T cells generated from day 8 HP cells, wherein the values are displayed as mean±SD, n=2, **p<0.01.

FIG. 5A is a diagram of time points at which cells were collected from single cell RNA-seq (scRNA-seq) analysis.

FIG. 5B shows UMAP layout of scRNA-seq data colored by cluster (left) or sample information (right).

FIG. 5C shows average gene expression levels and fractions of cells expressing the indicated genes in each cluster. AHE: arterial hemogenic endothelium. HE: hemogenic endothelium. EHT: endothelial-to-hematopoietic transition. HP: hematopoietic progenitor.

FIG. 5D shows UMAP layout expression of reflected genes.

FIG. 5E and FIG. 5F show average gene expression levels and fractions of cells expressing the genes in each cluster, wherein the fraction of eDLL413:Venus and DLL4Δi3/Δi3 (eDLL4i3 KO) are denoted in the upper plot. AHE: arterial hemogenic endothelium. HE: hemogenic endothelium. EHT: endothelial-to-hematopoietic transition. HP: hematopoietic progenitor.

FIG. 5G shows enrichment of selected Kyoto Encyclopedia of Genes and Genomes (KEGG) gene sets in each cluster.

FIG. 5H is a heat map showing the AUC Z-score of the top five most specific regulons in each cluster.

FIG. 6 shows the results of flow cytometric analysis of B cell markers from total floating HP cells at Day 8.

FIG. 7A is a heatmap showing differentially expressed genes (DEG, DEG definition: (log 2 fold change≥1 and adjusted p<0.05) in eDLL4i3:Venus⁺ compared to DLL4Δi3/Δi3 (eDLL4i3-KO) cells in ≥4 clusters. GAS5 is repressed in eDLL413:Venus⁺ compared to eDLL413 KO during EHT and HP stage. AHE: arterial hemogenic endothelium. HE: hemogenic endothelium. EHT: endothelial-to-hematopoietic transition. HP: hematopoietic progenitor.

FIG. 7B is a bar graph showing quantitative polymerase chain reaction (qPCR) analysis confirming upregulation of GAS5 expression in D5 CD31+ cells generated from DLL4Δi3/Δi3 (eDLL4i3-KO) hPSCs.

FIG. 8A is a diagram of differentiation in which on D5 of differentiation, cultures are transfected with GAS5 siRNA. On D8 of differentiation, HP isolated from cultures and analyzed for expression of GAS5 by qPCR (FIG. 8B) or T and B cell potential (FIGS. 9 and 10).

FIG. 8B is a bar graph showing GAS5 expression by qPCR from HP isolated from D8 of differentiation cultures shown in FIG. 8A.

FIG. 9A are representative dot plots showing the effect of GAS5 knockdown on T cell production from D8 HP. GAS5 knockdown restore impaired T cell production from DLL4Δi3/Δi3 (eDLL4i3-KO) hPSCs.

FIG. 9B are bar graphs showing the effect of GAS5 knockdown on T cell production from D8 HP. GAS5 knockdown restore impaired T cell production from DLL4Δi3/Δi3 (eDLL4i3-KO) hPSCs.

FIG. 10A are representative dot plots showing the effect of GAS5 knockdown on B cell production from D8 HP. DLL4Δi3/Δi3 (eDLL4i3-KO) hPSC generated significantly higher numbers of B cells. GAS5 knockdown had no effect on B cell production from unmodified wild type or DLL4Δi3/Δi3 hPSCs.

FIG. 10B are bar graphs showing the effect of GAS5 knockdown on B cell production from D8 HP. DLL4Δi3/Δi3 (eDLL4i3-KO) hPSC generated significantly higher numbers of B cells. GAS5 knockdown had no effect on B cell production from unmodified wild type or DLL4Δi3/Δi3 (eDLL4i3-KO) hPSCs.

FIG. 11A is a diagram of the experiments involving DLL4 and JAG1. WT or DLL4Δi3/Δi3 PSCs were differentiated for 8 days in defined conditions on plates precoated with collagen IV (control), DLL4-FC, or JAG1-Fc. On day 8 floating cells were collected and cultured on OP9-DLL4 or OP9 to produce T or B cells, respectively.

FIG. 11B shows representative dot plots demonstrating B cell production from WT and DLL4Δi3/Δi3 HPs.

FIG. 11C shows representative dot plots demonstrating T cell production from WT and DLL4Δi3/Δi3 HPs.

FIG. 11D and FIG. 11E show percentages and absolute counts of CD10+CD19+ cells in B cell cultures, respectively.

FIG. 11F and FIG. 11G show percentages and absolute counts of CD5+CD7+ cells in T cell cultures.

FIG. 11H and FIG. 11I show percentages and absolute counts of CD4+CD8+ cells in T cell cultures.

### DETAILED DESCRIPTION OF THE DISCLOSURE

This disclosure provides methods for producing arterial hemogenic endothelium and hematopoietic progenitor cells and differentiated progeny thereof, and cultures of arterial hemogenic endothelium, hematopoietic progenitor cells, or lymphoid cells, as well as pharmaceutical compositions comprising any of these cell cultures. Also provided herein are methods for promoting production of lymphoid cells from pluripotent stem cells by manipulating expression and activity of Notch ligands (DLL4 and JAG1) and GAS5.

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

The term “contacting” includes the physical contact of at least one substance to another substance or, for example, a cell.

As used herein, “treatment” refers to the clinical intervention made in response to a disease, disorder, or physiological condition of the subject or to which a subject can be susceptible. The aim of treatment includes the alleviation or prevention of symptoms, slowing or stopping the progression or worsening of a disease, disorder, or condition and/or the remission of the disease, disorder, or condition.

The terms “effective amount” or “therapeutically effective amount” refer to an amount sufficient to effect beneficial or desirable biological and/or clinical results. In other words, a “therapeutically effective” amount is an amount that will provide some alleviation, mitigation, or decrease in at least one clinical symptom in the subject.

The terms “express” or “expression” refer to transcription and translation of a nucleic acid coding sequence resulting in production of the encoded polypeptide. “Express” or “expression” also refers to antigens that are expressed on cell surfaces.

As used herein, the term “subject” refers to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The human subject can be of any age (e.g., an infant, child, or adult).

The term “construct” refers to an artificially-designed segment of DNA that can be used to incorporate genetic material into a target cell (e.g., an hPSC).

The term “sequence identity” refers to the number of identical or similar nucleotide bases on a comparison between a test and reference oligonucleotide or nucleotide sequence. Sequence identity can be determined by sequence alignment of a first nucleic acid sequence to identify regions of similarity or identity to second nucleic acid sequence. As described herein, sequence identity is generally determined by alignment to identify identical residues. Matches, mismatches, and gaps can be identified between compared sequences by techniques known in the art. Alternatively, sequence identity can be determined without taking into account gaps as the number of identical positions/length of the total aligned sequence×100. In one embodiment, the term “at least 90% sequence identity to” refers to percent identities from 90 to 100%, relative to the reference nucleotide sequence. Identity at a level of 90% or more is indicative of the fact that, assuming for exemplary purposes a test and reference polynucleotide sequence length of 100 nucleotides are compared, no more than 10% (i.e., 10 out of 100) of the nucleotides in the test oligonucleotide differ from those of the reference oligonucleotide. Differences are defined as nucleic acid substitutions, insertions, or deletions.

The term “genetically engineered” as used herein refers to cells that have been manipulated using biotechnology to change the genetic makeup of the cells, including the transfer of genes within and across species boundaries to produce improved or non-naturally occurring cells. A human pluripotent stem cell, hemogenic endothelium, megakaryocytes or NK cells that contains an exogenous, recombinant, synthetic, and/or otherwise modified polynucleotide is considered to be a genetically engineered cell and, thus, non-naturally occurring relative to any naturally occurring counterpart. In some cases, genetically engineered cells contain one or more recombinant nucleic acids. In other cases, genetically engineered cells contain one or more synthetic or genetically engineered nucleic acids (e.g., a nucleic acid containing at least one artificially created insertion, deletion, inversion, or substitution relative to the sequence found in its naturally occurring counterpart). Procedures for producing genetically engineered cells are generally known in the art, for example, as described in Sambrook et al, Molecular Cloning, A Laboratory Manual (Fourth Edition), Cold Spring Harbor Press, Cold Spring Harbor, N. Y. (2012) and Doudna et al., CRISPR-Cas, A Laboratory Manual, Cold Spring Harbor Press, Cold Spring Harbor, N.Y. (2016).

A genetically engineered cell can be a cell that has been modified using a gene editing technique. Gene editing refers to a type of genetic engineering in which DNA is inserted, deleted, modified, or replaced in the genome of a living cell. In contrast to other genetic engineering techniques that can randomly insert genetic material into a host genome, gene editing can target the insertions to site specific locations (e.g., AAVS1 alleles). Examples of gene editing techniques including, but are not limited to, restriction enzymes, zinc finger nucleases, TALENS, and CRISPR-Cas9.

A genetically engineered cell can be a stem cell (e.g., a human pluripotent stem cell) or any of their differentiated progeny cells (e.g., mesoderm cells, hemangioblast cells, hemogenic endothelium cells, hematopoietic progenitor cells, lymphomyeloid cells) that have been modified to express. Any of the cells described herein can be genetically engineered. In some embodiments, a genetically engineered cell refers to a cell that is differentiated from a cell that has been genetically engineered.

Human pluripotent stem cells (hPSCs), either embryonic or induced, provide access to the earliest stages of human development and offer a platform on which to derive a large number of hematopoietic progenitor cells or blood cells for cellular therapy and tissue engineering. Accordingly, the methods provided herein can comprise differentiating human pluripotent stem cells under conditions that promote differentiation of mesodermal cells into hemogenic endothelial cells, hematopoietic progenitor cells and lymphomyeloid cells.

Methods of differentiating hPSCs into progenitor mesoderm, hemogenic endothelium and hematopoietic progenitors are known in the art. In exemplary embodiments the culture medium to be used in any of the above-described differentiation methods comprises an IF9S medium, as described herein. In one embodiment, the IF9S medium to be used is the IF9S medium having the formulation set forth in Table 1. In a preferred embodiment, the cells are cultured on plates coated with Collagen IV, as described in Uenishi et al. (2014, Nat. Commun. 9: 1828) and U.S. Pat. No. 9,938,499.

In some embodiments, any of the above-referenced cells are cultured in a xeno-free cell culture medium. Of particular importance for clinical therapies is the absence of xenogenic materials in the derived cell populations, i.e., no non-human cells, cell fragments, sera, proteins, and the like. Preferably, the present invention arrives at xenogen-free differentiated cells by use of Collagen IV as a platform. In addition, the media disclosed herein are chemically defined and, in some embodiments, are made xeno-free, and incorporate human proteins, which can be produced using recombinant technology or derived from placenta or other human tissues in lieu of animal-derived proteins. In some embodiments, all proteins added to the medium are recombinant proteins.

As used herein, the term “mesoderm cell” refers to a cell having mesoderm-specific gene expression, capable of differentiating into a mesodermal lineage such as bone, muscle such as cardiac muscle, skeletal muscle, and smooth muscle (e.g., of the gut), connective tissue such as the dermis and cartilage, kidneys, the urogenital system, blood or hematopoietic cells, heart, and vasculature. Mesoderm-specific biomarkers include Brachyury (7). Culturing can take place on any appropriate surface (e.g., in two-dimensional or three-dimensional culture).

As used herein, the terms “chemically defined medium” and “chemically defined culture medium” also refer to a culture medium containing formulations of fully disclosed or identifiable ingredients, the precise quantities of which are known or identifiable and can be controlled individually. As such, a culture medium is not chemically defined if (1) the chemical and structural identity of all medium ingredients is not known, (2) the medium contains unknown quantities of any ingredients, or (3) both. Standardizing culture conditions by using a chemically defined culture medium minimizes the potential for lot-to-lot or batch-to-batch variations in materials to which the cells are exposed during cell culture. Accordingly, the effects of various differentiation factors are more predictable when added to cells and tissues cultured under chemically defined conditions.

As used herein, the term “serum-free” refers to cell culture materials that do not contain serum or serum replacement, or that contains essentially no serum or serum replacement. For example, an essentially serum-free medium can contain less than about 1%, 0.9%, 0.8%, 0.7%, 0.6%, 0.5%, 0.4%, 0.3%, 0.2%, or 0.1% serum. “Serum free” also refers to culture components free of serum obtained from animal (e.g., fetal bovine) blood or animal-derived materials, which is important to reduce or eliminate the potential for cross-species viral or prion transmission. For avoidance of doubt, serum-containing medium is not chemically defined.

As used herein, the term “hypoxic conditions” or “hypoxia” when referring to cell culture conditions is intended to mean 37° C., 5% O₂, and 5% CO₂).

As used herein, the term “normoxia conditions” or “normoxia” when referring to cell culture conditions is intended to mean 37° C., 21% O₂, and 5% CO₂).

Human pluripotent stem cells (e.g., human ESCs or iPS cells) can be cultured in the absence of a feeder layer (e.g., a fibroblast feeder layer), a conditioned medium, or a culture medium comprising poorly defined or undefined components. As used herein, “feeder-free” refers to culture conditions that are substantially free of a cell feeder layer. Cells grown under feeder-free conditions can be grown on a substrate, such as a chemically defined substrate, and/or grown as an adherent culture. Suitable chemically defined substrates include vitronectin.

As used herein, “differentiation potential” refers to the capability of undifferentiated stem cells to give rise to specialized cells.

As used herein, “pluripotent stem cells” appropriate for use according to a method of the invention are cells having the capacity to differentiate into cells of all three germ layers. Suitable pluripotent cells for use herein include human embryonic stem cells (hESCs) and human induced pluripotent stem (iPS) cells. As used herein, “embryonic stem cells” or “ESCs” mean a pluripotent cell or population of pluripotent cells derived from an inner cell mass of a blastocyst. See Thomson et al., Science 282:1145-1147 (1998). These cells can express Oct-4, SSEA-3, SSEA-4, TRA-1-60, and TRA-1-81. Pluripotent stem cells appear as compact colonies comprising cells having a high nucleus to cytoplasm ratio and prominent nucleolus. ESCs are commercially available from sources such as WiCell Research Institute (Madison, WI.).

As used herein, “induced pluripotent stem cells” or “iPS cells” refers to pluripotent cell or population of pluripotent cells that can vary with respect to their differentiated somatic cell of origin, that can vary with respect to a specific set of potency-determining factors and that can vary with respect to culture conditions used to isolate them, but nonetheless are substantially genetically identical to their respective differentiated somatic cell of origin and display characteristics similar to higher potency cells, such as ESCs, as described herein. See, e.g., Yu et al., Science 318:1917-1920 (2007).

Induced pluripotent stem cells exhibit morphological properties (e.g., round shape, large nucleoli, and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60, or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained from a post-natally.

The term “hemogenic endothelium” refers a subset of vascular endothelial cells that can be derived from PSCs and differentiated into hematopoietic progenitor cells. It is known that arterial fate acquisition of hemogenic endothelium cells, termed “arterial hemogenic endothelium” cells, increase the potential of these cells to differentiate to lymphoid cells. Hemogenic endothelium cells encompass both arterial and non-arterial hemogenic endothelium cells. Markers of hemogenic endothelium include, but are not limited to, CD144, CD34, CD31, CD44, ESAM, or APLNR. Markers of arterial hemogenic endothelium (AHE) include, but are not limited to, DLL4, CXCR4, or EFNB2. In some embodiments, the AHE cells express DLL4⁺/CXCR^{+/-}, while the HE cells express both AHE cells with DLL4⁺/CXCR^{+/-} and non-arterial HE cells expressing DLL4⁻/CXCR⁻.

The term “hematopoietic progenitor” refers to intermediate precursor cells that are derived from PSCs and hemogenic endothelium cells, and can differentiate into blood cells such as lymphoid cells (T cells, B cells, and Natural Killer cells) and myeloid cells. Markers of hematopoietic progenitor cells include, but are not limited to CD34, CD43, CD45, CD90, or CD123.

Methods for producing a hematopoietic progenitor cell can comprise culturing human pluripotent stem cells in a serum-free, albumin-free, chemically defined culture medium that promotes differentiation to mesoderm. In this manner, pluripotent stem cell-derived mesodermal cells are differentiated according to the HPC differentiation methods provided herein, thus producing pluripotent stem cell-derived HPCs.

Specifically, autologous hematopoietic stem cells (HSCs) can be produced from embryonic stem cells or induced pluripotent stem cells (iPSCs) from an individual. Although several cell culture methods have been attempted in the art they each have produced suboptimal results. Disclosed herein are more effective methods for producing arterial hemogenic endothelium (AHE), hematopoietic progenitor cells (HP cells or HPC), and lymphoid cells from pluripotent stem cells, and particularly iPSCs.

### Spheroid-Based Differentiation of Hematopoietic Progenitors

One aspect of the methods disclosed herein employs spheroid culture embodiments. The spheroid culture technique provides an appealing alternative to conventional monolayer culture because, inter alia, it can recreate biological signal pathways of cell-cell and cell-ECM interactions, which promotes cell proliferation and survival. There are several hematopoietic differentiation systems that have been reported earlier to generate arterial hemogenic endothelium and hematopoietic progenitors like coculture with feeder cells (Choi et al., 2011, Nat Protoc. 6:296-313), embryoid bodies (Daley, 2003, Annals of the New York academy of science 1:122-131; Lu et al., 2007, Nat. Methods 4:501-509), 2D culture (Uenishi et al., 2014, Stem Cell Reports 6:1073-1084) and forced expression of transcription factor (Sugimura et al., 2017, Nature 545:432-438). However due to undefined nature of feeder cells, large variability of embryoid body (EB) and requirement of large surface area in case of 2D, limits the scale up making this method unsuitable for future therapeutic manufacture. The spheroid culture technique can also alleviate monolayer culture challenges, such as the limited realization of in vivo multicellular microenvironments. As a result, differentiation potential, stemness, and inherent phenotypic traits can be more readily maintained. The differentiation potential of spheroids has been demonstrated for cancer immunotherapy research (Herter et al., 2017, Cancer Immunol. Immunother. 66: 129-140; Bocherit et al., 2020, Front. Immunol. 11: 603640), for example. Spheroid culture has also been shown to improve differentiation potential compared to monolayer culturing (Yamaguchi et al., 2014, BMC Biotechnology 14: 105) when used to differentiate endoderm from human embryonic stem cells (Bogacheva et al., 2021, Front. Cell Dev. Biol. 9 September). Despite these advantages, the spheroid culture technique has not been shown in the prior art to induce hematopoietic differentiation efficiently.

This disclosure provides methods for generating arterial hemogenic endothelium and hematopoietic progenitor cells using a 3D spheroid (75-150 mM). A low cost, specific medium is used to prepare the spheroid based on the “hanging drop method” in a short period of time (12-24 hrs). The hanging drop method to prepare the “Spheroid” is relatively low tech, easy to use and easily reproducible. The spheroid making medium is supplemented with appropriate components that further support the hematopoietic differentiation. Additionally, the percentage of arterial hemogenic endothelium (CD144+CD73−CD43−DLL4+CXCR4+) population around the sphere is higher than earlier reported methods (1-5% vs 10-20%). Furthermore, the generated hematopoietic progenitors around the sphere from AHE are 70-95% positive for CD34 and CD43. Finally, enhanced production of T and NK cells from arterial hemogenic endothelium (CD7+CD5+˜72-80%; CD8a+CD4˜55.4-60%; CD8a+CD8b+˜40-50% and CD56+CD16+˜30-40%) can also be achieved with this method.

As disclosed herein, these deficiencies in spheroid culture technique have been overcome using a hanging drop culture method in conjunction with the spheroid culture technique. This method has been used for a variety of purposes since its initial disclosure in the art (see, Harrison et al., 1907, Proc. Soc. Exp. Bio. Med. 4: 140-143). Starting with a monolayer cell culture, the cells are produced as a suspension and diluted with culture media to achieve the appropriate cell density. The cell suspension is then distributed into the wells of a mini-tray using a suitable multistep or multichannel pipette. The mini-tray is covered with a lid and turned completely upside down. Surface tension would keep the cell suspension droplets fixed to the mini-tray on the reversed surface. As used herein, spheroids are formed as droplets owing to simultaneous action of surface tension and gravitational force. This permits dissociated single cell suspensions to be aggregated and for spheroids to be fabricated as droplets. Spheroid size can be controlled using the volume of the drop or density of cell suspension.

This technique can form circular spheroids with a narrow size distribution with a 10% to 15% variation coefficient. In comparison, the spheroid growth in non-adherent surface culture methods has a 40% to 60% variation coefficient. Advantageously, a considerable quantity of spheroids can be produced readily by multichannel pipetting and can be harvested readily by scraping the lids of culture dishes.

Prior unsuccessful attempts to adapt the hanging drop technique for hematopoietic differentiation (Cerdan et al., 2007, Curr. Protoc. Stem Cell Biol. Chapter 1, Unit 1D.2) and cardiomyocyte differentiation (Yoon et al., 2006, Differentiation 74: 149-159) from clumps of undifferentiated hESCs were the result, inter alia, of there being a considerable amount of size variability between EB as it was formed from chunks of hESC and not from the single-cell suspension. The spheroid formed from chunks/clumps was not only non-uniform in size but also did not adopt a circular shape. The droplets were too small and too flat and hence stuck to the plate after droplet formation and showed a delay in hematopoietic differentiation. These methods were also less efficient, taking 15 days to achieve 10% of the cells to express CD34 CD45 biomarkers for hematopoietic progenitor cells. Thus, this approach was not feasible for current and future cell therapy applications.

As disclosed herein, hanging drop cell culture and arterial hemogenic endothelium and hematopoietic progenitor cell differentiation methods are used in single-cell suspension of human PSC (ESC/iPSC) in a droplet.

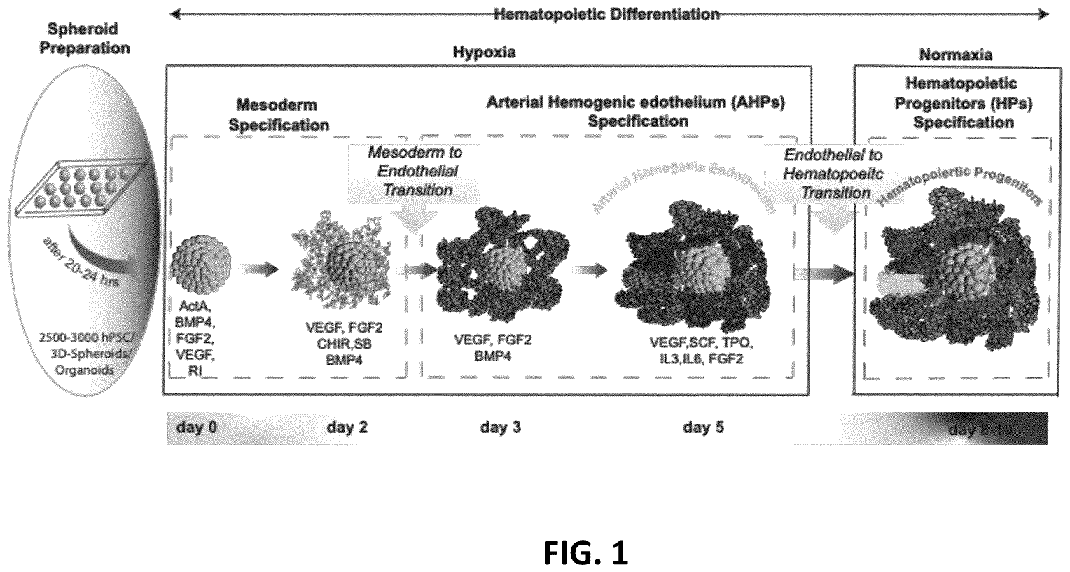

Moreover, efficient and effective hematopoietic progenitor production from iPSCs required differentiation to occur in conjunction with arterial hemogenic endothelium (AHE), which has been understood to be an essential prerequisite to HSC differentiation (Uenishi et al., 2014, Stem Cell Reports 3: 1073-1084). These biological features of normal HSC development are adapted as a component of the spheroid culture hanging drop methods disclosed herein. FIG. 1 of the Drawings describes an embodiment of the spheroid culture hanging drop method to differentiate hPSCs into hematopoietic progenitors (HPs).

As set forth herein, spheroid cultures are prepared in serum-free spheroid medium. “Spheroid medium” is defined as medium in which the cells were suspended to form a spheroid droplet. Spheroid size can be controlled using the volume of the drop or density of cell suspension. The volume of the drop can range from 30-40 μL while the density of cells in the drop can range from 2,500-3,500 cells. In the practice of these methods 2,500-3,500 cells, in particular embodiments 3,000 cells, are dispersed into each droplet.

In certain embodiments, the spheroid medium contains 40% ES-Cult M3120 (containing 2.6% methylcellulose in Iscove's Modified Dulbecco's medium; Stem Cell Technologies), 25% Dulbecco's Modified Eagle medium (Corning), 25% Ham's F-12 Medium (Corning), 10% BIT 9500 supplements comprising bovine serum albumin, insulin, and transferrin (Stem Cell Technologies), GlutaMAX (1/100 dilution from 200 mM L-alanyl-L-glutamine dipeptide in 0.85% NaCl stock or 2 mM working concentration; Invitrogen), non-essential Amino Acids (NEAA) (1/100 dilution from 10 mM stock; Life Technologies), 100 μM monothioglycerol (MTG), and 100 μg/ml ascorbic acid. The recipes of each ingredient and medium are shown in Tables 4-8. The cell-comprising spheroids are incubated for about 20-24 hours in cell-sustaining temperature and humidity conditions for about 20-24 hours in cell-sustaining temperature and humidity conditions.

As provided herein is a method to produce hematopoietic progenitor cells from the cell-comprising spheroids described above, comprising the following steps:
(a) harvesting cell-comprising spheroids with a buffer medium that is phosphate-buffered saline or iPSC culture medium by centrifugating, and resuspending the spheroids in IF9S medium supplemented with BMP4, VEGF, FGF, Activin A, and ROCK inhibitor (at concentration ranges shown in Table 2). About 140-160 spheroids are plated per 50-60 cm² surface area of plates coated with collagen IV. The ingredients of basal IF9S medium are provided in Table 1,
(b) culturing the plated spheroids/cells under hypoxic conditions (5% O₂, 5% CO₂),
(c) after 46-50 hours or on day 2 (48 hours), the medium is changed to IF9S supplemented with VEGF, FGF, BMP4, CHIR99021, and SB-43152 (at concentration ranges shown in Table 2),
(d) after 24 hours or on day 3, the medium is replaced with IF9S supplemented with VEGF, FGF, and BMP4 (a concentration ranges shown in Table 2); wherein the spheroids remain in the hypoxic incubator,
(e) after 48 hours or on day 5, the medium is changed to IF9S medium supplemented with SCF, VEGF, TPO, IL6, FGF, and IL3 on day 5 (concentration range shown in Table 2) and the spheroids are transferred to normoxia incubator with 5% CO₂ to produce arterial hemogenic endothelium cells,
(f) after 48 hours or on day 7, more IF9S medium supplemented with SCF, VEGF, TPO, IL6, FGF, and IL3 is added to produce hematopoietic progenitors,
(g) after two to four days, harvesting floating hematopoietic progenitor cells from the culture medium or dislodging hematopoietic progenitor cells attached to the surface.

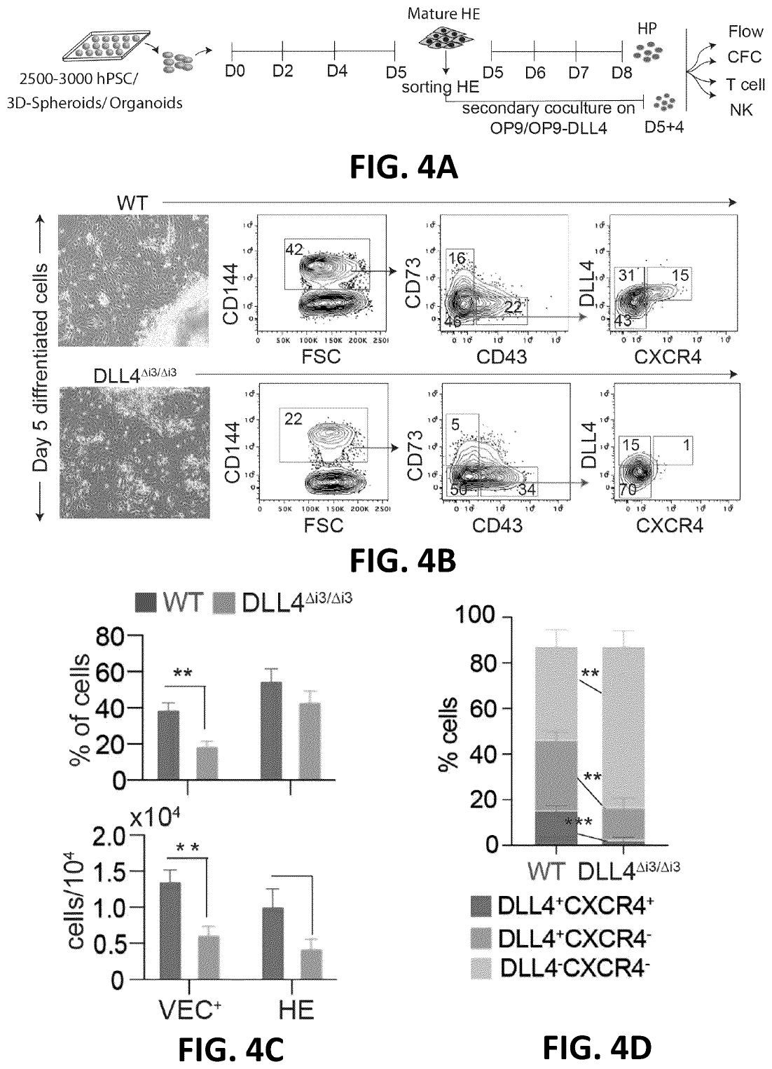

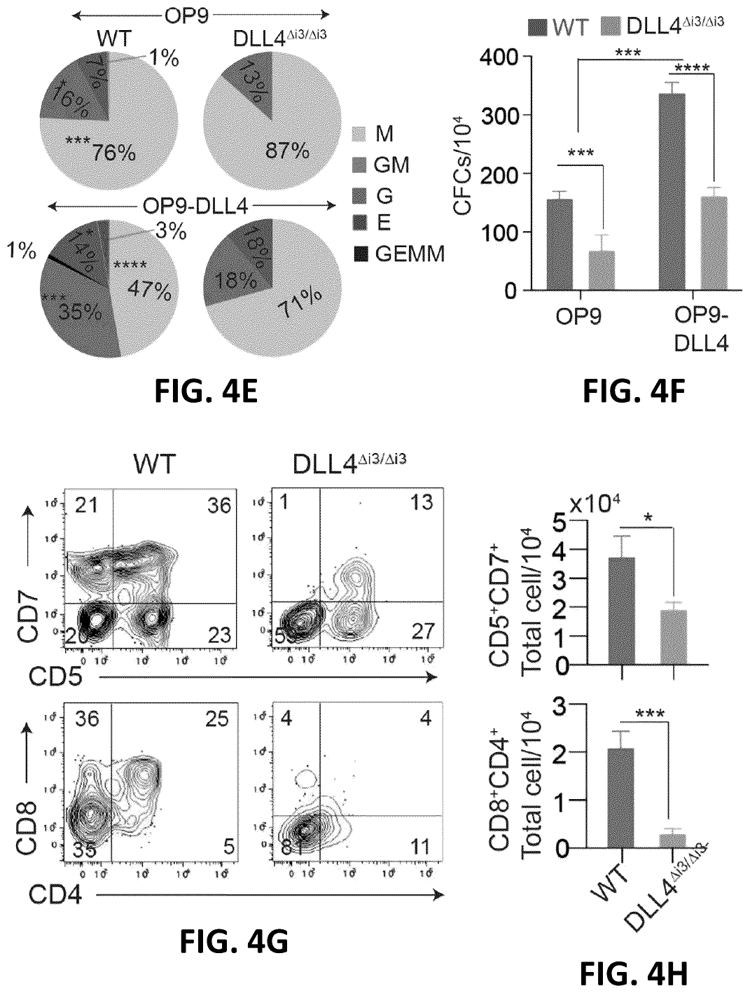

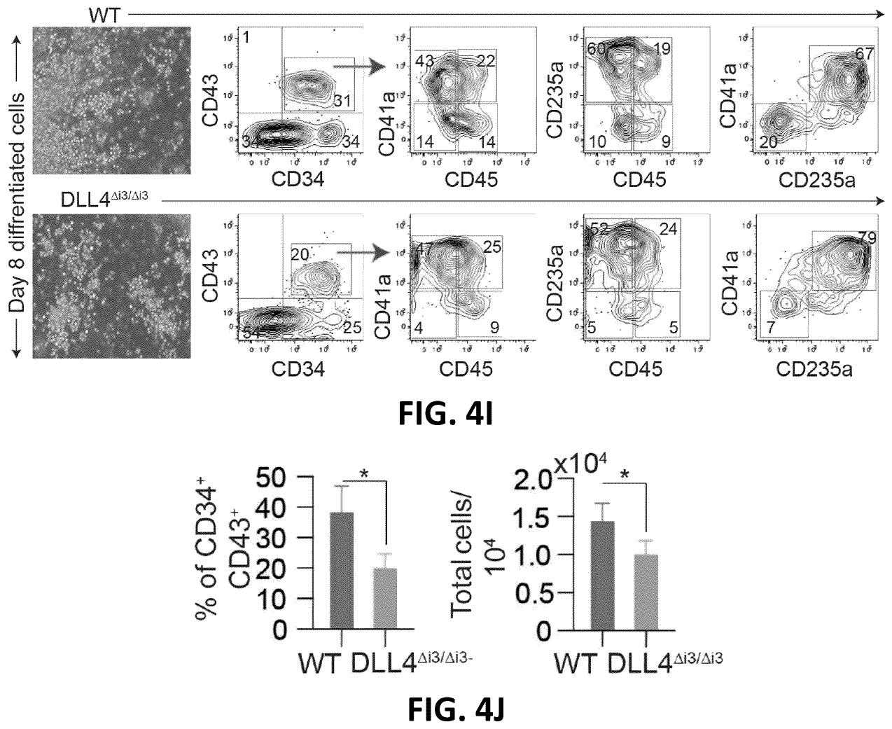

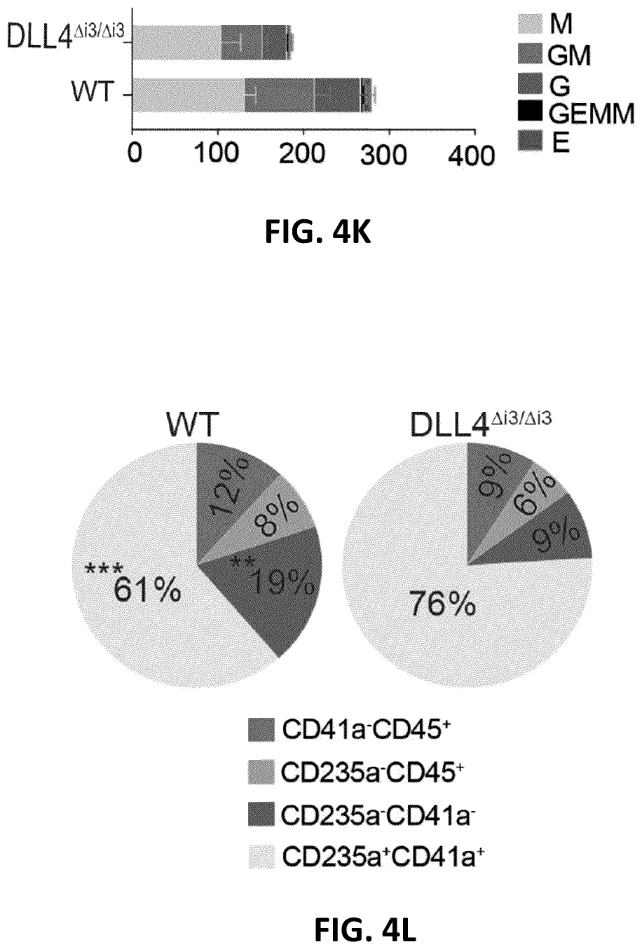

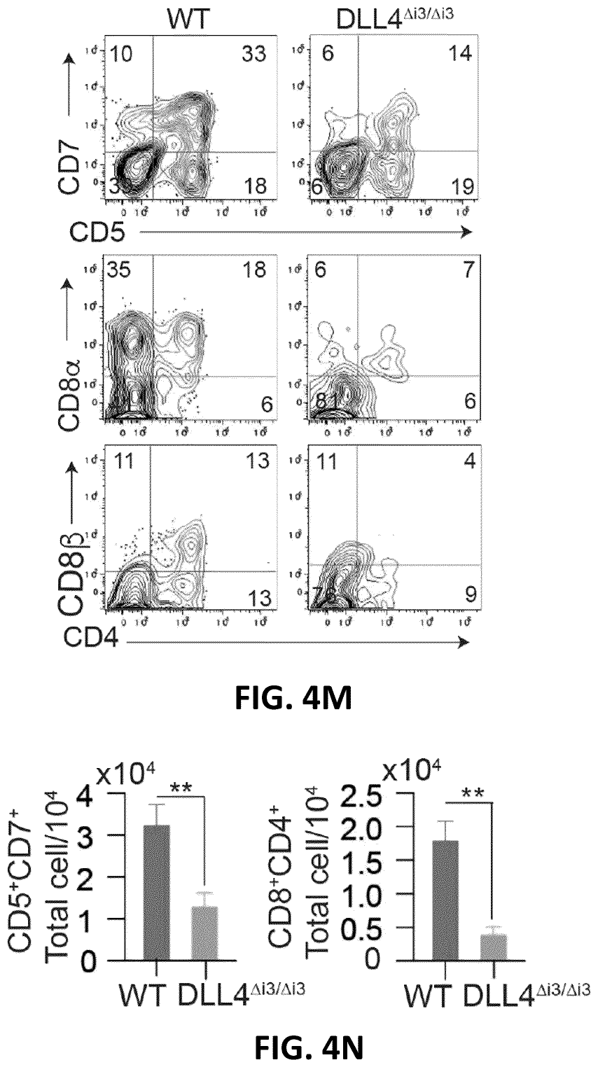

The floating HPs are collected to differentiate further into T cells or B cells as described below, or for further studies. According to the methods disclosed herein, D5 differentiated PSC cultures produce at least 40% of hemogenic endothelium (HE) with at least 50% of the hemogenic endothelium cells are AHE cells having a biomarker phenotype of CD144⁺CD73⁻CD43⁻DLL4⁺CXCR^{+/-} AHE cells and having T lymphoid potential (FIG. 4D). In other words, the described method increases production of AHE cells with T lymphoid potential.

### Reporter of Arterial-Type Hemogenic Endothelium (AHE)-Specific Hematopoietic Progenitor Cells

Also disclosed herein are DLL4 enhancer reporter constructs that are introduced into the PSCs. During vascular development, arterial fate is specified following induction of DLL4 expression by VEGF (Lawson et al., 2001, Development 128: 3675-3683). DLL4 expression is initiated by signaling through a highly conserved VEGF-responsive artery-specific enhancer located within the third intron of DLL4 (eDLL413) which is controlled by ETS, SOXF and RBPJ factors (Sacilotto et al., 2013, Proc Natl Acad Sci USA 110: 11893-11898; Wythe et al., 2013, Dev Cell 26: 45-58). The eDLL413 reporter is used for lineage tracing that maps differentiation of arterial-type hemogenic endothelium (AHE)-specific hematopoietic progenitor cells from PSC expressing the reporter. Thus, this method is capable of establishing direct progenitor-progeny links between arterial specification and definitive hematopoiesis. In addition, molecular profiling results show that DLL4⁺ hemogenic endothelium (HE), in contrast to DLL4⁻ HE, have an arterial molecular signature that is identified by increased expression of NOTCH1, NOTCH4, JAG2, HEY1, HEY2, SOX17 and EFNB2 (Uenishi et al., 2018m Nat. Commun. 9: 1828). Using a transgenic eDLL413 reporter construct, combined with gene editing by CRISPR/Cas9 and stem cell technology disclosed herein is a real-time AHE-specific map of hematopoietic differentiation.

Gene interaction analysis identifies the third intron of DLL4 gene as a highly conserved VEGF-responsive artery-specific enhancer, which is controlled by ETS, SOXF, and RBJPJ factors (Sacilotto et al., 2013, Proc Natl Acad Sci USA 110: 11893-11898; Wythe et al., 2013, Dev Cell 26: 45-58). Thus, the third intron of DLL4 gene was selected to be introduced into the cells. In certain embodiments, the eDLL413 reporter construct comprises of DLL4 third intron enhancer gene (nucleotide at position +1352 to +2108 of gene ID: 54567 was used, SEQ ID NO: 16) in conjunction with β-globin promoters and VENUS reporter gene. The construct was cloned into the AAVS1-SA-2A-Puro donor plasmid. The donor plasmid was introduced into the AAVS1 locus in the cells using CRISPR/CAS9 system with sgRNA targeting the AAVS1 sequence. The sequence of DLL4 third intron enhancer gene is as below:

(SEQ ID NO: 16)
CTCGCTCCGCCACCACAGGGGGGCGACACGGCGCAGCGCCGAAAGAGTT
AATCTGTTCTAGGCGGGGGAAGTGCGGGCTTGGGGGTGGGAGGCAGGAC
GCTTAGCTTGGCCTGGAGCTGCGCCCCGCGCTGGACGCTCGGATTCCGC
TCGCTGCCTGGACTCAGAGCACAATTGCGTTTCCTGCGGGTTATTTTTG
GCGTGGGAACGCGGGGAGTACGGCGGTGAGAAAGGCTGAAGCTGCCAGC
GCCGCTGACGGGCCCCTTCCTGTATTTTACACCTTTCGCGAATTCCGCT
CCTTTGGAAAGGGAATAATGGCTTTGGGATGTTGTTCTGACACAGAGGA
AAAGGATATTTCAGCAGCACAACAATTCTCACTTTGAAAAGGAAAAAAG
AAAACCATTACCCACCTCTGGAGGCAGAACCCCTGAATGGGCACCAAAG
GACCCCCTGCTCCCAGGGTCCTCTCTAGCCTGGGGAGCTTTTCTTTCTT
TTTCTCTTTTTTCCATTTTGACCTCTTTTCCTCTTTCCCCTCCCTATCT
GCCTCCAAGACCCTGGGATATCTTAACATCCTTCTATTGTCCCCTTTTT
GAATACTATCAGGCCCCCTGCACATGCACACACGTAGGGCAGCTACGTA
GCGGGGCTTTGGGTCCCTCTGGCCTGTTCTTGCTGGCAGGCGGGGGTCA
TCTGGATAACTGGGCTGATTGGTTGGCTGATCACCATCATCACAGCCAA
GAAGGACATTGGCCAGCCGTCA

### T Cell and Natural Killer (NK) Cells Differentiation

T cell differentiation was performed according to earlier reported protocol reported in Kumar et al. (2020, Bio-Protocol 10: ARTN e3675); Kumar et al. (2019, J Immunol 202: 770-776), and U.S. Pat. No. 11,345,895B2, the disclosures of which are incorporated herein by reference. Briefly, in certain embodiments, floating hematopoietic cells at Day 9 (D9), D4+5 (cells are sorted on day 4 and cultured for additional 5 days) and D5+4 (cells are sorted on day 5 and cultured for additional 4 days), which were produced by the spheroid-based differentiation method described above, were collected, strained, and cultured on OP9-DLL4 in minimum essential medium eagle alpha modification (aMEM) (Gibco) with 20% FBS (HyClone), 10 ng/ml SCF, 5 ng/ml FLT3L and 5 ng/ml IL-7 (PeproTech) for 3 weeks. Other concentration ranges of these ingredients are described in Table 3. Floating cells were passaged weekly onto fresh OP9-DLL4 cells. Note that OP9 macrophage-derived embryonic stem cell line do not express Notch ligand DLL4 while OP9-DLL4 is transfected with DLL4 gene to induce Notch signaling activity required for T cell differentiation.

Similarly, for NK cell differentiation, floating HPs from D9, D4+5 and D5+4 were collected, strained, and cultured on OP9 DLL4 in aMEM with 20% FBS containing 100 ng/ml FLT3L, 40 ng/ml SCF and 5 ng/ml IL7 for 1 week. After a week, cells were cultured in aMEM with 20% FBS containing 10 ng/ml of IL15 and 5 ng/ml of IL2. Cells were analyzed for the different lymphoid markers by flow cytometry. Other concentration ranges of the ingredients are described in Table 3.

According to the methods disclosed herein, T cells and NK cells can be produced by AHE having the phenotypic characteristics of ˜72-80% CD7⁺CD5⁺; ˜ 55.4060% CD8a⁻CD4⁺; 40-50% CD8a⁺CD8b⁺; and ˜30-40% CD56⁺CD16⁺.

Generation of T cells from spheroid-derived hematopoietic cells, can be performed using any T cell differentiation methods from hematopoietic progenitor cells that have been described elsewhere, for examples, methods described in U.S. Patent Publication Numbers US20220403326A1, US20180305664A1, which are incorporated herein by reference.

### B Cell Differentiation

B cell differentiation was performed according to conventional protocols. Briefly, in certain embodiments, floating D8/D9 hematopoietic cells, which were produced by the spheroid-based differentiation method described above, were collected and cultured on OP9 in minimum essential medium (aMEM, Gibco) with 20% FBS (HyClone), 5 ng/ml FLT3L (Peprotech) and 5 ng/ml IL7 (Peprotech) for 4 weeks. Other concentration ranges of these ingredients are described in Table 3.

Generation of B cells from spheroid-derived hematopoietic cells, can be performed using any B cell differentiation methods from hematopoietic progenitor cells that have been described in the art, for example, Carpenter et al. (2011, Blood 117(15): 4008-11), Vodyanik et al. (2005, Blood 105(2): 617-26), Richardson et al. (2021, STAR Protoc. 2(2): 1000420), the disclosures of which are incorporated herein by reference.

Methods to Increase Lymphoid Cell Production by Controlling GAS5 Expression and NOTCH Activity with Notch Ligands (DLL4 and JAG1)

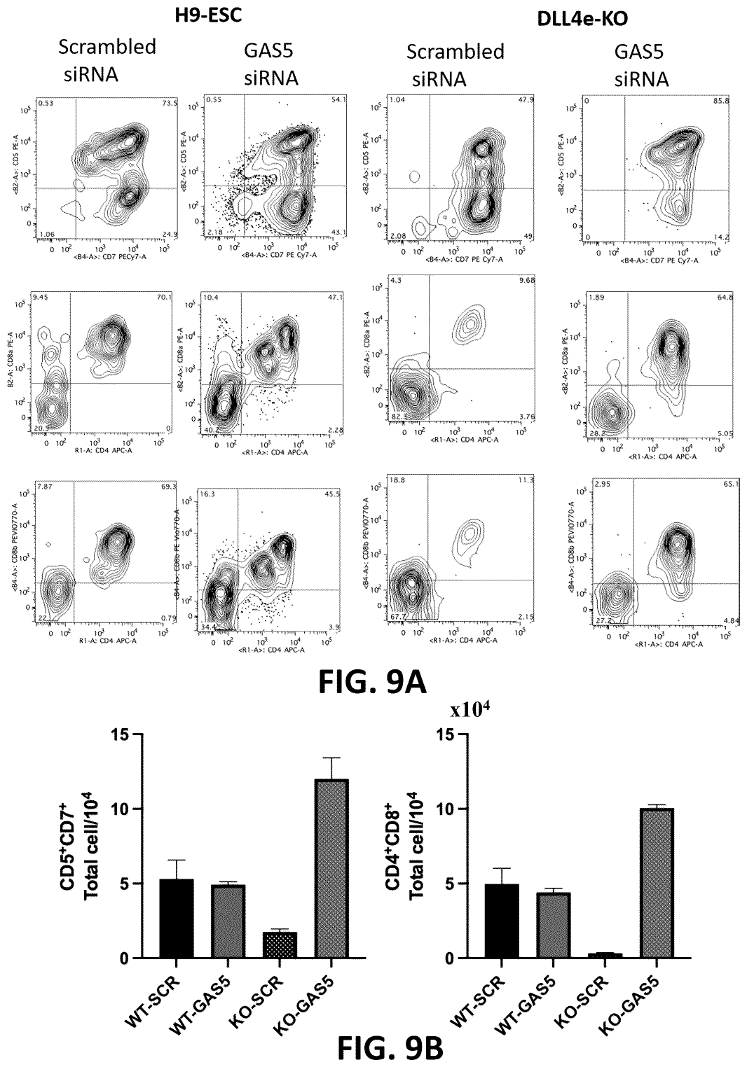

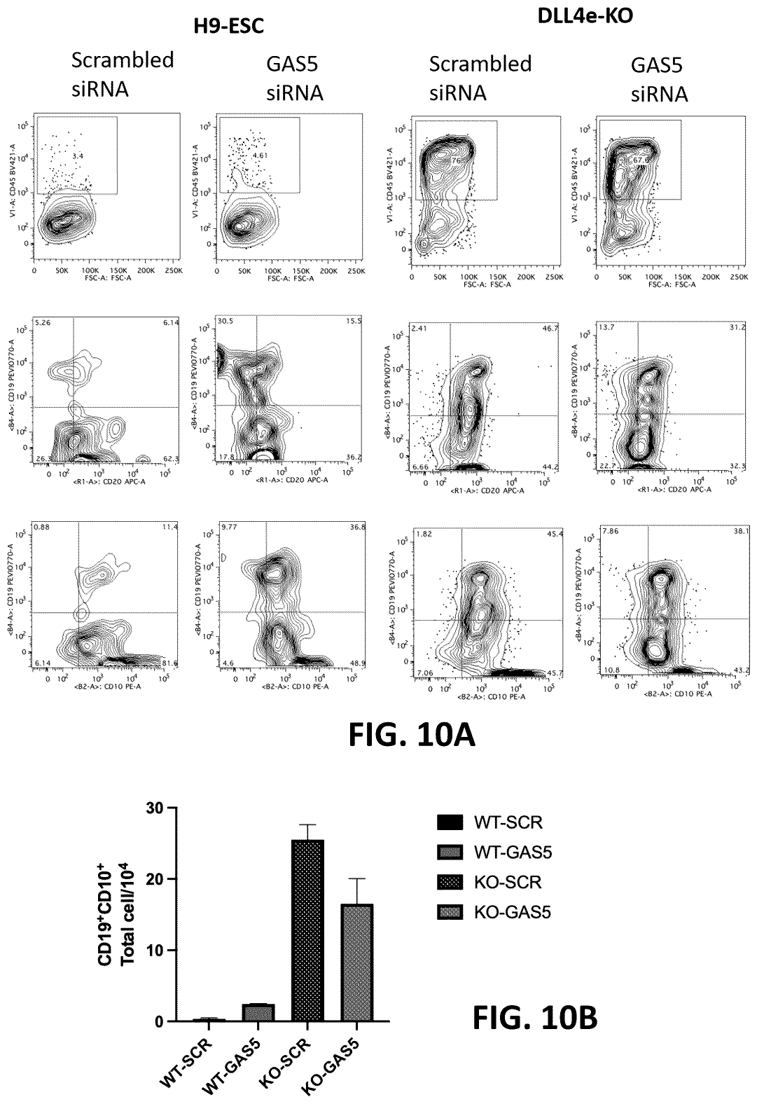

Provided herein are also methods to increase differentiation of lymphoid cells from PSCs. Experiments demonstrated herein show that reduction of DLL4 expression by knocking out the artery-specific enhancer within intron 3 of DLL4 (eDLL4i3 KO) in PSCs produce less DLL4⁺CXCR^{+/-} AHE cells (1-18%) compared to wild-type PSCs (10-45%) on day 5 of differentiation using the methods described above (see FIG. 4D). The day 5 hemogenic endothelium cells derived from eDLL4i3KO PSCs had a significant decrease in T cells and increase in B cells differentiation potential (FIG. 9B and FIG. 10B respectively). However, when GAS5 expression was reduced on eDLL413KO PSCs-derived day 5 hemogenic endothelium cells, T cell potential was restored and T cell production was surprisingly greater than the production from wild-type DLL4 and GAS5 expressing PSCs (FIG. 9A and FIG. 9B). In addition, eDLL413 KO PSCs cultured on JAG1 coated plates from day 0 at a concentration ranging from 0.5-1.5 μg/cm² had increased B cell differentiation potential compared to eDLL4i3KO and wild type PSCs grown on collagen coated plates. Critically, growing eDLL413 KO PSCs in the presence of JAG1 restored T cell differentiation potential and surprisingly increased T cell production compared to eDLL4i3KO and wild type PSCs grown on collagen coated plates.

Reduction of DLL4 expression and activity in PSCs, in combination with suppression of GAS5 expression in hemogenic endothelium cells, or with JAG1 presence from PSC to hematopoietic progenitor stage, can be applied in any method of differentiation from PSCs into AHE/HE cells or HP cells. Other methods of AHE differentiation from PSCs have been described in US20210261923A1 and US20220306988A1, which are fully incorporated by reference herein.

Reduction of DLL4 expression and activity in PSCs alone, or in combination with JAG1 presence from PSC to hematopoietic progenitor stage, can generally be expected to promote B cell production in any method of differentiation from PSCs into AHE/HE cells or HP cells.

### Methods to Control Expression and Activity of DLL4 and GAS5

Methods of gene editing well known in the art can be used to control expression of DLL4 and GAS5. In certain embodiments, DLL4 expression is attenuated by KO of DLL4 third intron enhancer sequence using CRISPR/Cas9. To generate eDLL413 enhancer KO (eDLL4i3 KO) H9 ESC line, two single guide RNAs were designed in CRISPR design tool (Synthego). sgRNA recognition sequence and PAM were:

SEQ ID NO: 8
5′-AGTTAATCTGTTCTAGGCGG-3′ (sgRNA1), PAM: GGG
and
SEQ ID NO: 9
5′-CGGGGGTCATCTGGATAACT-3′ (sgRNA2), PAM: GGG.

Clonally amplified cells were picked and screened by genomic PCR for the 632 bp deletion in wildtype DLL4 enhancer allele using the following primers:

(forward)
SEQ ID NO: 10
GGTACCTTCTCGCTCATCATC,
(reverse)
SEQ ID NO: 11
AGTGGTCATTGCGCTTCTT.

The deleted 632 base pairs were nucleotides at position +1414 to +2045 of wild type DLL4 enhancer gene (gene ID: 54567) and shown below:

(SEQ ID NO: 17)
CGGGGGAAGTGCGGGCTTGGGGGGGGAGGCAGGACGCTTAGCTTGGCCT
GGAGCTGCGCCCCGCGCTGGACGCTCGGATTCCGCTCGCTGCCTGGACT
CAGAGCACAATTGCGTTTCCTGCGGGTTATTTTTGGCGTGGGAACGCGG
GGAGTACGGCGGTGAGAAAGGCTGAAGCTGCCAGCGCCGCTGACGGGCC
CCTTCCTGTATTTTACACCTTTCGCGAATTCCGCTCCTTTGGAAAGGGA
ATAATGGCTTTGGGATGTTGTTCTGACACAGAGGAAAAGGATATTTCAG
CAGCACAACAATTCTCACTTTGAAAAGGAAAAAAGAAAACCATTACCCA
CCTCTGGAGGCAGAACCCCTGAATGGGCACCAAAGGACCCCCTGCTCCC
AGGGTCCTCTCTAGCCTGGGGAGCTTTTCTTTCTTTTTCTCTTTTTTCC
ATTTTGACCTCTTTTCCTCTTTCCCCTCCCTATCTGCCTCCAAGACCCT
GGGATATCTTAACATCCTTCTATTGTCCCCTTTTTGAATACTATCAGGC
CCCCTGCACATGCACACACGTAGGGCAGCTACGTAGCGGGGCTTTGGGT
CCCTCTGGCCTGTTCTTGCTGGCAGGCGGGGGTCATCTGGATA.

In certain embodiments, GAS5 non-coding RNA is inhibited by siRNA targeting GAS5 sequences GAUGGAGUCUCAUGGCACA—SEQ ID NO: 12, UGGAUGACUUGCUUGGGUA—SEQ ID NO: 13, AGGUAUGGAGAGUCGGCUU—SEQ ID NO: 14, AGGCAGACCUGUUAUCCUA—SEQ ID NO: 15. Examples of other gene editing techniques including, but are not limited to, restriction enzymes, zinc finger nucleases, and TALENs. KO of DLL4 expression can also be achieved by deletion of exon sequences using CRISPR/Cas9, TALEN and other nucleases inhibiting DLL4 expression using siRNA, shRNA, or CRISPR-mediated inhibition.

It has been contemplated but not demonstrated that DLL4 activity can also be suppressed by using antibody competitively binds to Notch receptors and competes with DLL4 binding or antibodies that bind to DLL4 and block its interaction with Notch receptors (Koga et al., 2014, Methods Mol Biol. 1187: 335-42).

### Pharmaceutical Formulations and Methods of Treatment

Administration of a therapeutically effective amount of hematopoietic progenitor cells provided herein into the recipient subject is generally effected using methods well known in the art, and usually involves directly injecting or otherwise introducing a therapeutically effective dose of hematopoietic progenitor cells, or lymphoid and myeloid cells derived from thereof into the subject using clinical tools known to those skilled in the art (e.g., U.S. Pat. Nos. 6,447,765; 6,383,481; 6,143,292; and 6,326,198). For example, introduction of hematopoietic progenitor cells produced by the methods set forth herein or otherwise can be injected locally or systemically via intravascular administration, such as intravenous, intramuscular, or intra-arterial administration, intraperitoneal administration, and the like. Cells can be injected into an infusion bag (e.g., Fenwal infusion bag (Fenwal, Inc.)) using sterile syringes or other sterile transfer mechanisms. The cells can then be immediately infused via IV administration over a period of time, such as 15 minutes, into a free flow IV line into the patient. In some embodiments, additional reagents such as buffers or salts are provided to the recipient subject concurrently with the cells.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

TABLE 1
Ingredients of basal IF9S medium

| IF9S medium | Amount | Unit/L |
|---|---|---|
| Inorganic Salts | | |
| Calcium Chloride | 99.3 | mg |
| Cupric Sulfate•5H2O | 0.00125 | mg |
| Ferrous Sulfate•7H2O | 0.417 | mg |
| Magnesium Chloride•6H2O | 28.8 | mg |
| Magnesium Sulfate (anhydrous) | 48.835 | mg |
| Potassium Chloride | 277 | mg |
| Potassium Nitrate | 0.038 | mg |
| Sodium Bicarbonate | 2100 | mg |
| Sodium Chloride | 6052 | mg |
| Sodium Phosphate Dibasic (anhydrous) | 71.02 | mg |
| Sodium Phosphate Monobasic (anhydrous) | 54.5 | mg |
| sodium selenite | 0.017 | mg |
| Zinc Sulfate•7H2O | 0.4315 | mg |
| Amino Acids | | |
| glycine | 26.255 | mg |
| L-alanyl-L-glutamine dipeptide | 434.4 | mg |
| L-alanine | 25.9 | mg |
| L-Arginine•HCl | 160.7 | mg |
| L-asparagine | 21.705 | mg |
| L-Aspartic acid | 34.95 | mg |
| L-Cysteine•HCl•H2O | 17.5 | mg |
| L-Cystine•2HCl | 45.62 | mg |
| L-Glutamic Acid | 59.55 | mg |
| L-Glutamine | 365 | mg |
| L-Histidine•HCl•H2O | 31.48 | mg |
| L-Isoleucine | 54.47 | mg |
| L-Leucine | 59.05 | mg |
| L-Lysine•HCl | 91.25 | mg |
| L-Methionine | 17.24 | mg |
| L-Phenylalanine | 35.48 | mg |
| L-proline | 48.75 | mg |
| L-Serine | 36.75 | mg |
| L-Threonine | 53.45 | mg |
| L-Tryptophan | 9.02 | mg |
| L-Tyrosine•2Na•2H2O | 55.785 | mg |
| L-Valine | 52.85 | mg |
| Vitamins | | |
| D-Biotin | 0.01015 | mg |
| Choline Chloride | 8.98 | mg |
| Folic Acid | 2.66 | mg |
| holo-transferrin | 10.7 | mg |
| myo-Inositol | 12.6 | mg |
| Niacinamide | 2.0185 | mg |
| D-Pantothenic Acid (hemicalcium) | 2.24 | mg |
| Pyridoxal•HCl | 2 | mg |
| Pyridoxine•HCl | 0.031 | mg |
| Riboflavin | 0.219 | mg |
| thiamine | 2.17 | mg |
| Vitamin B12 | 0.6865 | mg |
| Other | | |
| D-Glucose | 3151 | mg |
| HEPES | 2979 | mg |
| Hypoxanthine | 2.04 | mg |
| Phenol Red•Na | 8.65 | mg |
| Putrescine•HCl | 0.0805 | mg |
| DL-Thioctic Acid | 0.105 | mg |
| Pyruvic Acid•Na | 110 | mg |
| Thymidine | 0.365 | mg |
| 1-thioglycerol | 40 | ul |
| polyvinyl alcohol | 10 | g |
| insulin | 20 | mg |
| L-ascorbic acid 2-phosphate magnesium salt | 64 | mg |
| Lipids | | |
| arachidonic acid | 0.004 | mg |
| cholesterol | 0.44 | mg |
| DL-alpha-Tocopherol Acetate | 0.14 | mg |
| Linoleic Acid | 0.0956 | mg |
| Linolenic Acid | 0.02 | mg |
| Myristic Acid | 0.02 | mg |
| Oleic Acid | 0.02 | mg |
| Palmitic Acid | 0.02 | mg |
| Palmitoleic Acid | 0.02 | mg |
| Pluronic F-68 | 180 | mg |
| Stearic Acid | 0.02 | mg |
| Tween-80 | 4.4 | mg |

TABLE 2
Quantity of ingredients supplemented in IF9S medium.

| Supplement Ingredients | Amount | Unit |
|---|---|---|
| Bone morphogenetic protein 4 (BMP4) | 10-50¹ | ng/ml |
| | 10-20² | |
| Vascular endothelial growth factor (VEGF) | 25-50 | ng/ml |
| Fibroblast growth factor (FGF) | 10-50^{a} | ng/ml |
| | 10-20^{b} | |
| Activin A | 10-15 | ng/ml |
| Rho Kinase (ROCK) inhibitor | 1-10 | μM |
| Glycogen synthase kinase 3 inhibitor | 1-3 | μM |
| CHIR99021 | | |
| Activin receptor-like kinase receptors | 3-5 | μM |
| inhibitor SB-431542 | | |
| Stem cell factor (SCF) | 25-50 | ng/ml |
| Thrombopoietin (TPO) | 25-50 | ng/ml |
| Interleukin 6 (IL6) | 25-50 | ng/ml |
| Interleukin 3 (IL3) | 5-10 | ng/ml |

¹for step (a)
²for step (c) and (d)
^{a}for step (a), (c), and (d)
^{b}for step (e) and (f)

TABLE 3
Quantity of ingredients supplemented
in minimum essential medium (aMEM).

| Supplement Ingredients | Amount | Unit |
|---|---|---|
| Stem cell factor (SCF) | 10-25¹ | ng/ml |
| | 30-40² | ng/ml |
| FLT-3L | 5-10^{1,3} | ng/ml |
| | 60-100² | ng/ml |
| Interleukin 7 (IL7) | 5-10 | ng/ml |
| Interleukin 15 (IL15) | 10-15 | ng/ml |
| Interleukin 2 (IL2) | 5-10 | ng/ml |

¹for T cell differentiation
²for NK cell differentiation
³for B cell differentiation

TABLE 4
Ingredients of Dulbecco's Modified Eagle's
medium as provided by vendor (Corning).
Dulbecco's Modified Eagle's medium

| Components | Unit (mg/L) |
|---|---|
| Inorganic Salts | |
| CaCl₂ (anhydrous) | 200 |
| Fe(NO₃)₃•9H₂O | 0.1 |
| KCl | 400 |
| MgSO₄ (anhydrous) | 97.7 |
| NaCl | 6400 |
| NaH₂PO₄•H₂O | 125 |
| NaHCO₃ | 3700 |
| Amino Acids | |
| L-Arginine•HCl | 84.00 |
| L-Cystine•2HCl | 62.57 |
| L-Glutamine | 584.00 |
| Glycine | 30.00 |
| L-Histidine•HCl•H₂O | 42.00 |
| L-Isoleucine | 104.80 |
| L-Leucine | 104.80 |
| L-Lysine•HCl | 146.20 |
| L-Methionine | 30.00 |
| L-Phenylalanine | 66.00 |
| L-Serine | 42.00 |
| L-Threonine | 95.20 |
| L-Tryptophan | 16.00 |
| L-Tyrosine•2Na•2H₂O | 103.79 |
| L-Valine | 94.00 |
| Vitamins | |
| D-Calcium pantothenate | 4.00 |
| Choline chloride | 4.00 |
| Folic acid | 4.00 |
| i-Inositol | 7.20 |
| Nicotinamide | 4.00 |
| Pyridoxine•HCl | 4.00 |
| Riboflavin | 0.40 |
| Thiamine•HCl | 4.00 |
| Other | |
| D-Glucose | 4500.00 |
| Phenol red•Na | 15.00 |
| Sodium pyruvate | 110.00 |
| HEPES | 5958.00 |

TABLE 5
Ingredients of Ham's F-12 medium as provided by vendor (Corning).
Ham's F-12

| Components | Unit (mg/L) |
|---|---|
| Inorganic Salts | |
| CaCl₂ (anhydrous) | 33.3 |
| FeSO₄•7H₂O | 0.834 |
| KCl | 223.6 |
| CuSO₄ (anhydrous) | 0.00016 |
| MgSO₄ (anhydrous) | 72.2 |
| NaCl | 7600 |
| Na₂HPO₄ (anhydrous) | 142 |
| NaHCO₃ | 1176 |
| ZnSO₄•7H₂O | 0.863 |
| Amino Acids | |
| L-Alanine | 8.9 |
| L-Arginine•HCI | 211 |
| L-Asparagine•H₂O | 15 |
| L-Aspartic acid | 13.3 |
| L-Cysteine•HCl•H₂O | 35.12 |
| L-Glutamic acid | 14.7 |
| L-glutamine | 146.2 |
| Glycine | 7.5 |
| L-Histidine•HCl•H₂O | 20.96 |
| L-Isoleucine | 3.94 |
| L-Leucine | 13.1 |
| L-Lysine•HCl | 36.5 |
| L-Methionine | 4.48 |
| L-Phenylalanine | 4.96 |
| L-Proline | 34.5 |
| L-Serine | 10.5 |
| L-Threonine | 11.9 |
| L-Tryptophan | 2.04 |
| L-Tyrosine•2Na•2H₂O | 7.84 |
| L-Valine | 11.7 |
| Vitamins | |
| Biotin | 0.0073 |
| D-Ca-Pantothenate | 0.48 |
| Choline Chloride | 13.96 |
| Folic Acid | 1.3 |
| I-Inositol | 18.02 |
| Nicotinamide | 0.037 |
| Pyridoxine•HCl | 0.062 |
| Riboflavin | 0.038 |
| Thiamine•HCl | 0.34 |
| Vitamin B12 | 1.36 |
| Other | |
| Dextrose | 1802 |
| Hypoxanthine, Sodium | 4.77 |
| DL-Thioctic (lipoic) acid | 0.21 |
| Methyl Lineoleate | 0.088 |
| Phenol Red, Sodium | 1.2 |
| Putrescine•2HCl | 0.16 |
| Sodium Pyruvate | 110 |
| Thymidine | 0.73 |

TABLE 6
Ingredients of Iscove's Modified Dulbecco's Medium
as provided by vendor (Stem Cell Technologies).
Iscove's Modified Dulbecco's Medium

| Components | |
|---|---|
| Inorganic Salts | Unit (g/L) |
| Calcium Chloride•2H₂O | 0.219 |
| Magnesium Sulfate | 0.09767 |
| Potassium Chloride | 0.33 |
| Potassium Nitrate | 0.000076 |
| Sodium Chloride | 4.505 |
| Sodium Phosphate Monobasic | 0.109 |
| Sodium Selenite | 0.000017 |
| | g/L |
| Amino Acids | |
| L-Alanine | 0.025 |
| L-Arginine•HCl | 0.084 |
| L-Asparagine•H₂O | 0.0284 |
| L-Aspartic Acid | 0.03 |
| L-Cystine•2HCl | 0.09124 |
| L-Glutamic Acid | 0.075 |
| L-Glutamine | 0.584 |
| Glycine | 0.03 |
| L-Histidine•HCl•H₂O | 0.042 |
| L-Isoleucine | 0.105 |
| L-Leucine | 0.105 |
| L-Lysine•HCl | 0.146 |
| L-Methionine | 0.03 |
| L-Phenylalanine | 0.066 |
| L-Proline | 0.04 |
| L-Serine | 0.042 |
| L-Threonine | 0.095 |
| L-Tryptophan | 0.016 |
| L-Tyrosine•2Na•2H₂O | 0.10379 |
| L-Valine | 0.094 |
| Vitamins | |
| D-Biotin | 0.000013 |
| Choline Chloride | 0.004 |
| Folic Acid | 0.004 |
| myo-Inositol | 0.0072 |
| Niacinamide | 0.004 |
| D-Pantothenic Acid (hemicalcium) | 0.004 |
| Pyridoxine•HCl | 0.004 |
| Riboflavin | 0.0004 |
| Thiamine•HCl | 0.004 |
| Vitamin B-12 | 0.000013 |
| Other | |
| D-Glucose | 4.5 |
| HEPES | 5.958 |
| Phenol Red•Na | 0.016 |
| Pyruvic Acid•Na | 0.11 |
| Sodium Bicarbonate | 3.024 |

TABLE 7
Ingredients of Non-essential Amino Acids as
provided by the vendor (Life Technologies).

| | | | |
|---|---|---|---|
| | | Concentration | |
| Components | Molecular Weight | (mg/L) | mM |

Amino Acids

| | | | |
|---|---|---|---|
| Glycine | 75 | 750 | 10 |
| L-Alanine | 89 | 890 | 10 |
| L-Asparagine | 132 | 1320 | 10 |
| L-Aspartic acid | 133 | 1330 | 10 |
| L-Glutamic Acid | 147 | 1470 | 10 |
| L-Proline | 115 | 1150 | 10 |
| L-Serine | 105 | 1050 | 10 |

TABLE 8
Ingredients of MEMα as provided
by the vendor (Life Technologies).

| | | | |
|---|---|---|---|
| | | Concen- | |
| | Molecular | tration | |
| Components | Weight | (mg/L) | mM |

Amino Acids

| | | | |
|---|---|---|---|
| Glycine | 75 | 50 | 0.6666667 |
| L-Alanine | 89 | 25 | 0.28089887 |
| L-Arginine hydrochloride | 211 | 105 | 0.49763033 |
| L-Asparagine-H2O | 150 | 50 | 0.33333334 |
| L-Aspartic acid | 133 | 30 | 0.22556391 |
| L-Cysteine hydrochloride-H2O | 176 | 100 | 0.5681818 |
| L-Cystine 2HCl | 313 | 31 | 0.09904154 |
| L-Glutamic Acid | 147 | 75 | 0.5102041 |
| L-Glutamine | 146 | 292 | 2 |
| L-Histidine | 155 | 31 | 0.2 |
| L-Isoleucine | 131 | 52.4 | 0.4 |
| L-Leucine | 131 | 52 | 0.39694658 |
| L-Lysine | 183 | 73 | 0.3989071 |
| L-Methionine | 149 | 15 | 0.10067114 |
| L-Phenylalanine | 165 | 32 | 0.19393939 |
| L-Proline | 115 | 40 | 0.3478261 |
| L-Serine | 105 | 25 | 0.23809524 |
| L-Threonine | 119 | 48 | 0.40336135 |
| L-Tryptophan | 204 | 10 | 0.04901961 |
| L-Tyrosine disodium salt | 225 | 52 | 0.23111111 |
| L-Valine | 117 | 46 | 0.3931624 |

Vitamins

| | | | |
|---|---|---|---|
| Ascorbic Acid | 176 | 50 | 0.2840909 |
| Biotin | 244 | 0.1 | 4.10E−04 |
| Choline chloride | 140 | 1 | 0.007142857 |
| D-Calcium pantothenate | 477 | 1 | 0.002096436 |
| Folic Acid | 441 | 1 | 0.002267574 |
| Niacinamide | 122 | 1 | 0.008196721 |
| Pyridoxal hydrochloride | 204 | 1 | 0.004901961 |
| Riboflavin | 376 | 0.1 | 2.66E−04 |
| Thiamine hydrochloride | 337 | 1 | 0.002967359 |
| Vitamin B12 | 1355 | 1.36 | 0.00100369 |
| i-Inositol | 180 | 2 | 0.011111111 |

Inorganic Salts

| | | | |
|---|---|---|---|
| Calcium Chloride (CaCl2) | 111 | 200 | 1.8018018 |
| (anhyd.) | | | |
| Magnesium Sulfate (MgSO4) | 120 | 97.67 | 0.8139166 |
| (anhyd.) | | | |
| Potassium Chloride (KCl) | 75 | 400 | 5.3333335 |
| Sodium Bicarbonate (NaHCO3) | 84 | 2200 | 26.190475 |
| Sodium Chloride (NaCl) | 58 | 6800 | 117.24138 |
| Sodium Phosphate monobasic | 138 | 140 | 1.0144928 |
| (NaH2PO4—H2O) | | | |

Ribonucleosides

| | | | |
|---|---|---|---|
| Adenosine | 267 | 10 | 0.037453182 |
| Cytidine | 243 | 10 | 0.041152265 |
| Guanosine | 283 | 10 | 0.03533569 |
| Uridine | 244 | 10 | 0.040983606 |

Deoxyribonucleosides

| | | | |
|---|---|---|---|
| 2′Deoxyadenosine | 251 | 10 | 0.03984064 |
| 2′Deoxycytidine HCl | 264 | 11 | 0.041666668 |
| 2′Deoxyguanosine | 267 | 10 | 0.037453182 |
| Thymidine | 242 | 10 | 0.041322313 |

Other Components

| | | | |
|---|---|---|---|
| D-Glucose (Dextrose) | 180 | 1000 | 5.5555553 |
| Lipoic Acid | 206 | 0.2 | 9.71E−04 |
| Phenol Red | 376.4 | 10 | 0.026567481 |
| Sodium Pyruvate | 110 | 110 | 1 |

### EXAMPLES

The Examples set forth herein incorporate and rely on certain experimental and preparatory methods and techniques preformed as exemplified herein.

### Materials and Methods

Cell Culture. hPSCs lines H9 ESCs (WA09) and BM-iPSCs (BM9) from WiCell were cultured on Matrigel-coated plate (BD Biosciences, USA) in mTeSR media (WiCell) with daily media changes. Cells were split weekly by dissociation with 0.5 mM EDTA (Sigma, USA). hESC cultures were visualized daily by phase contrast microscopy.

Construction of vectors and generation of DLL4 enhancer reporter hESC line. To target the donor cassette to the AAVS1 locus in H9 human ESCs, a human eDLL4i3 757 bp sequence (position +1352 to 2108 of gene ID: 54567), in conjunction with the β-globin promoters and VENUS (a yellow fluorescent protein; see, Nagai et al., 2002, Nature Biotechnol. 20: 87-90) was cloned into the AAVS1-SA-2A-Puro vector. CRISPR/Cas9 nuclease system was used to target at the AAVS1 locus. sgRNA recognition sequence and PAM were 5′-GGGGCCACTAGGGACAGGAT-3′ SEQ ID NO: 1, PAM:TGG (Synthego). 2×10⁶ single cells dissociated from H9 hESCs were resuspended in Nucleofector Solution™ (Lonza) and electroporated with 1 μg of Cas9 protein (PNA Bio), 1 μg of sgRNA and 1 μg of AAVS1-SA-2A-Puro-DLL4 enhancer-VENUS donor plasmid. Cells were subsequently plated at a low density on Matrigel coated 6-well plate in mTeSR1 medium supplemented with CloneR (Stemcell). The culture were maintained for 5 days and then selected with 0.5 μg/mL Puromycin (Sigma) for up to 7-10 days until colonies form. Puromycin-resistant colonies were picked and expanded.

PCR screening for DLL4 enhancer reporter clones. Genomic DNA of puromycin-resistant colonies was extracted using ZR-96 Quick-gDNA kit (Zymo research). PCR primers for screening correctly targeted cells were:

(forward)
SEQ ID NO: 2
TTCCCAGGCAGGTCCTGCTTTCTCTGA
and
(reverse)
SEQ ID NO: 3
GTGGGCTTGTACTCGGTCAT.

PCR amplification products were separated on 1% agarose gels. The expected size of fragment from correctly targeted DLL4 enhancer cells was 959 bp.

Southern blot analysis. Further confirmation of gene targeting was performed by Southern blot analysis. Genomic DNA of wild-type and DLL4 enhancer reporter hESCs were extracted using FlexiGene DNA kit (QIAGEN). 10 μg of genomic DNA from each clone were digested with EcoRV (for VENUS probe) or ApaL1 (for 3′ probe) overnight and separated by gel electrophoresis on a 0.7% agarose gel for 6 hours. The gel was depurinated, denatured, neutralized, and blotted by capillary transfer with 20×SSC on a nylon membrane (Amersham). The 3′ probe was generated using the following primers:

(forward)
SEQ ID NO: 4
TCTAACAGGTACCATGTGGGGTT
and
(reverse)
SEQ ID NO: 5
ATCTAGGTAGCCACAGGAGGC,

using VENUS cassette probe

(forward)
SEQ ID NO: 6
ATATAAGCTTATGGTGAGCAAGGGCGAGGAGCT
and
(reverse)
SEQ ID NO: 7
ATGCCTTGTACAGCTCGTCCATGCCGAGA..

The DNA membrane was UV crosslinked and hybridized overnight with digoxigenin (DIG)-labelled probes according to the manufacturer's instructions (Roche).

Generation of eDLL4i3 KO H9 hESC lines. To generate eDLL413 KO H9 ESC line, two single guide RNAs were designed in CRISPR design tool (Synthego). sgRNA recognition sequence and PAM were

SEQ ID NO: 8
5′-AGTTAATCTGTTCTAGGCGG-3′ (sgRNA1), PAM: GGG
and
SEQ ID NO: 9
5′-CGGGGGTCATCTGGATAACT-3′ (sgRNA2), PAM: GGG.

Clonally amplified cells were picked and screened by genomic PCR for the 632 bp deletion in wildtype DLL4 enhancer allele using the following primers:

(forward)
SEQ ID NO: 10
GGTACCTTCTCGCTCATCATC,
(reverse)
SEQ ID NO: 11
AGTGGTCATTGCGCTTCTT.

Hematopoietic differentiation of hPSCs. hPSCs were differentiated into hematopoietic lineages using a novel spheroid-based differentiation protocol. On day −1, hPSCs were singularized and resuspended in a spheroid medium. The spheroid medium contained 40% ES-Cult M3120 (containing 2.6% Methylcellulose in Iscove's Modified Dulbecco's medium; Stem Cell Technologies), 50% Dulbecco's Modified Eagle's medium/Hams F-12 50/50 Mix (Corning), 10% BIT 9500 supplements (comprising bovine serum albumen, insulin, and transferrin; Stem Cell Technologies), GlutaMAX (1/100 dilution of 200 mM stock; Invitrogen), Non-essential Amino Acids (NEAA) (1/100 dilution of 10 mM stock; Life Technologies), 100 μM monothioglycerol (MTG), and 100 μg/ml ascorbic acid. Small droplets of 30 μl were made on square dishes such that each spheroid contained about 3000 cells and the plate was closed and placed in an inverted position overnight in the incubator so that the cells can aggregate in the drop by the hanging drop method. The following day, spheroids were harvested with phosphate-buffered saline or iPSC medium, centrifuged, and resuspended in IF9S medium supplemented with BMP4 (50 ng/ml), VEGF (50 ng/ml), FGF (50 ng/ml), Activin A (15 ng/ml) and ROCK inhibitor (1 μM). About 140-160 spheroids were plated on six well plates (57.6 cm² surface area) or 100 mm dish (57 cm² surface area) coated with collagen IV (at a concentration of about 0.5 g/cm²), recombinant DLL4 fused with Fc protein (DLL4-Fc) (at a concentration of about 0.5 μg/cm²), or JAG1-Fc (at a concentration of 0.5 μg/cm²) and cultured in hypoxia (5% 02, 5% CO₂). On Day 2 or after 48 hours, the media was changed to IF9S supplemented with VEGF (50 ng/ml), FGF (50 ng/ml), BMP4 (10 ng/ml), CHIR99021 (1 μM), SB-431542 (3 μM) and placed back in the hypoxia incubator. On Day 3, the medium was replaced with IF9S medium supplemented with VEGF (50 ng/ml), FGF (50 ng/ml), BMP4 (10 ng/ml) and placed back in the hypoxia incubator. For some studies, the plates were taken out on Day 4 and magnetic-activated cell sorting (MACS) enriched for CD31+ hemogenic endothelium and replated on either OP9/OP9 DLL4 plates or on collagen IV or DLL4Fc (DLL4 fused with Fc portion of immunoglobin to activate NOTCH signaling) coated plates. On Day 5, the medium was replaced with IF9S medium supplemented with SCF (50 ng/ml), VEGF (50 ng/ml), TPO (50 ng/ml), IL6 (50 ng/ml), FGF (10 ng/ml), IL3 (10 ng/ml) and the plates were placed in normoxia incubator with 5% CO₂. For some studies the culture plates on Day 5 were sorted by fluorescence activated cell sorting (FACS) to isolate D5 hemogenic endothelium subsets for further studies. On Day 7, additional IF9S medium supplemented SCF, VEGF (50 ng/ml), TPO (50 ng/ml), IL6 (50 ng/ml), FGF (10 ng/ml), IL3 (10 ng/ml) was added. Hematopoietic progenitors (HPs) were collected on D8 or D10 for further studies.

Hematopoietic Colony Forming Cell (CFC) assay. Hematopoietic colony forming unit assay was conducted in serum-containing H4435 Methocult (Stem Cell Technologies) according to manufacturer instruction and the hematopoietic colonies were analyzed at day 12. The colony forming cell (CFC) assay was used to study the proliferation and differentiation pattern of hematopoietic progenitors by their ability to form colonies in a semisolid medium.

Lymphoid cell (T cell, B Cell, and NK cell) differentiation. T cell differentiation was performed according to earlier reported protocol (Kumar et al., 2020, Bio-Protocol 10: ARTN e3675; Kumar et al., 2019, J. Immunol. 202: 770-776). Briefly, floating hematopoietic cells at Day 9, Day 4+4 and Day 5+4 were collected, strained, and cultured on OP9-DLL4 in minimum essential Eagle medium with alpha modification (MEMα, Gibco) with 20% FBS (HyClone), 10 ng/ml SCF, 5 ng/ml FLT3L and 5 ng/ml of IL-7 (PeproTech) for 3 weeks. Floating cells were passaged weekly onto fresh OP9-DLL4 cells. Floating cells at 21 days were analyzed by flow cytometry for CD5, CD7, CD4, CD8a, CD8b, CD3 and TCRab expression. For B cell differentiation, D8 floating HPs were collected and cultured on OP9 in minimum essential medium (aMEM, Gibco) with 20% FBS (HyClone), 5 ng/ml FLT3L (Peprotech) and 5 ng/ml IL7 (Peprotech) for 3 weeks. For NK cell differentiation, floating HPs from D9, D4+4 and D5+4 were collected, strained, and cultured on OP9 DLL4 in aMEM with 20% FBS containing 100 ng/ml FLT3L, 40 ng/ml SCF and 5 ng/ml IL7 for 1 week. After a week, cells were cultured in a MEM with 20% FBS containing 10 ng/ml of IL15 and 5 ng/ml of IL2. Cells were analyzed for the different lymphoid markers by flow cytometry.

Single Cell RNA-seq and data processing. Single cell library preparation, sequencing, and analysis were conducted by Singulomics Corporation (https://singulomics.com/, Bronx, NY). Cryopreserved, viable single cell suspensions were thawed, washed, resuspended in cell culture media with 0.04% BSA, and counted. Dead cells were removed from the single cell suspensions using the Dead Cell Removal Kit (Miltenyi Biotec, Sam Diego, CA) and cells were counted again. Viable cell suspensions were then loaded into the Chromium Controller (10× Genomics, Pleasanton, CA) to generate gel beads-in-emulsion (GEM) with each GEM containing a single cell as well as barcoded oligonucleotides. Six thousand cells per sample were targeted for capture. Next, the GEMs were placed in the SimpliAmp 96-well Thermal Cycler (Thermo Fisher Scientific, Wilmington, DE) and reverse transcription was performed in each GEM (GEM-RT). After the reaction, complementary cDNA was amplified and cleaned using Silane DynaBeads (10× Genomics, Pleasanton, CA) and the SPRIselect Reagent kit.

GAS5 siRNA studies For knockdown of GAS5 long non-coding (Inc) RNA, cells were transfected with 100 nM of GAS5 siRNA control pool (Target Sequences-GAUGGAGUCUCAUGGCACA—SEQ ID NO: 12, UGGAUGACUUGCUUGGGUA—SEQ ID NO: 13, AGGUAUGGAGAGUCGGCUU—SEQ ID NO: 14, AGGCAGACCUGUUAUCCUA—SEQ ID NO: 15, Dharmacon) or Scramble negative control siRNA (Target Sequence-UGGUUUACAUGUCGACUAA—SEQ ID NO: 18, Dharmacon) on day 5 of differentiation using Lipofectamine RNAiMAX (ThermoFisher). HPs were collected on Day 8 of differentiation for T cell and B cell differentiation and for qPCR analysis.

Statistical analysis. Data were analyzed using GraphPad Prism version 9 (GraphPad Software Inc.) and Microsoft Excel (Microsoft Corporation). Tests for statistical significance are listed with each experiment; these included two-sided Student's t-tests for paired analyses and one-way ANOVAs, and two-way ANOVAs for experiments with multiple comparisons of variables or grouped variables, accompanied by the Tukey and Sidak post-hoc test, as inferred to be most appropriate by the software.

### Example 1: Generation and Validation of eDLL4i3:VENUS Reporter hESC Line

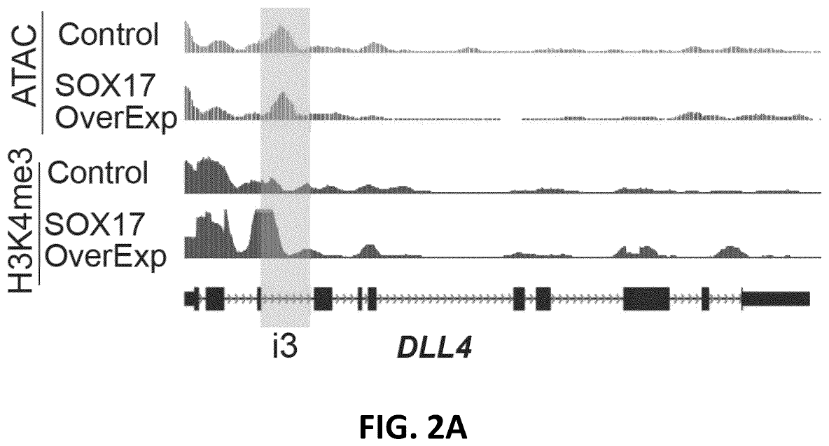

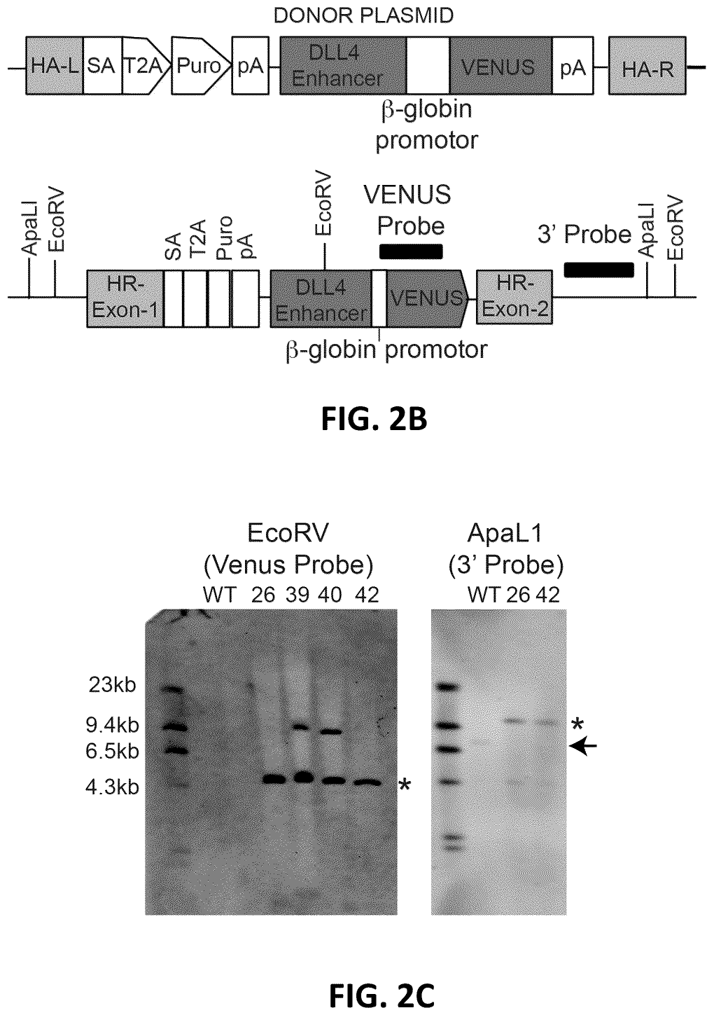

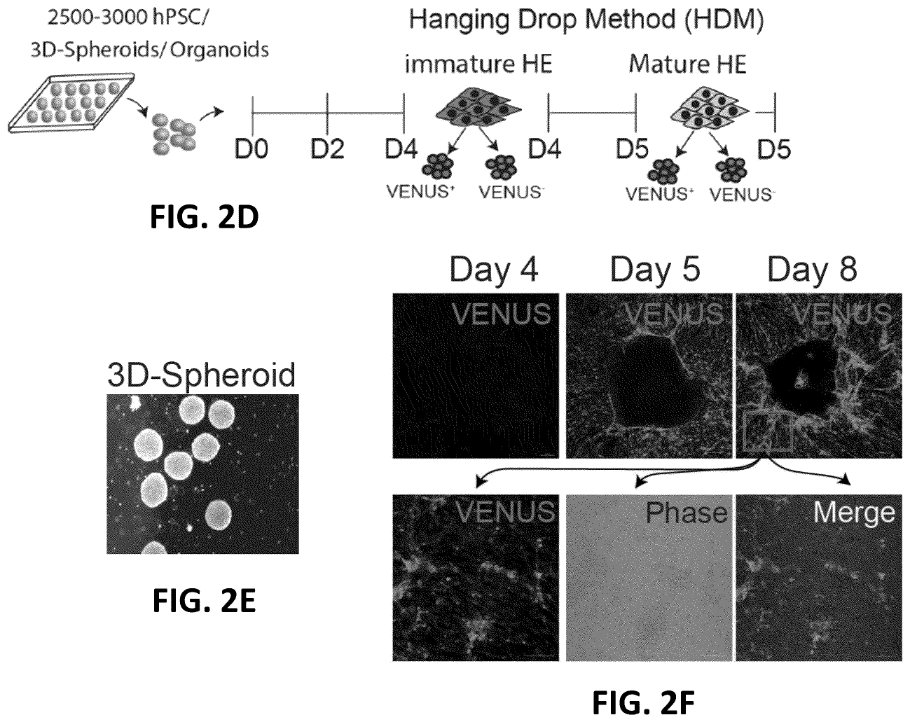

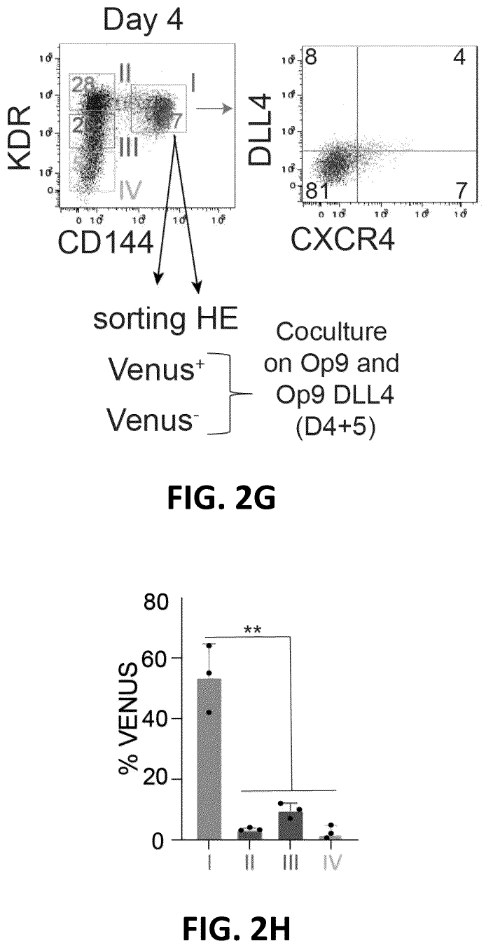

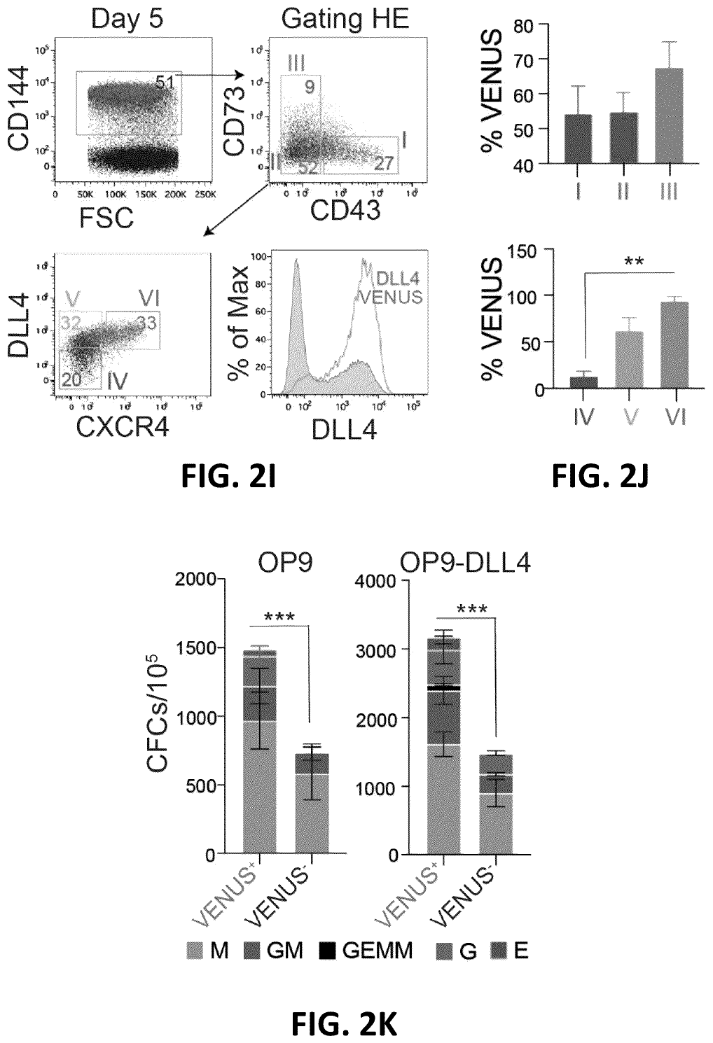

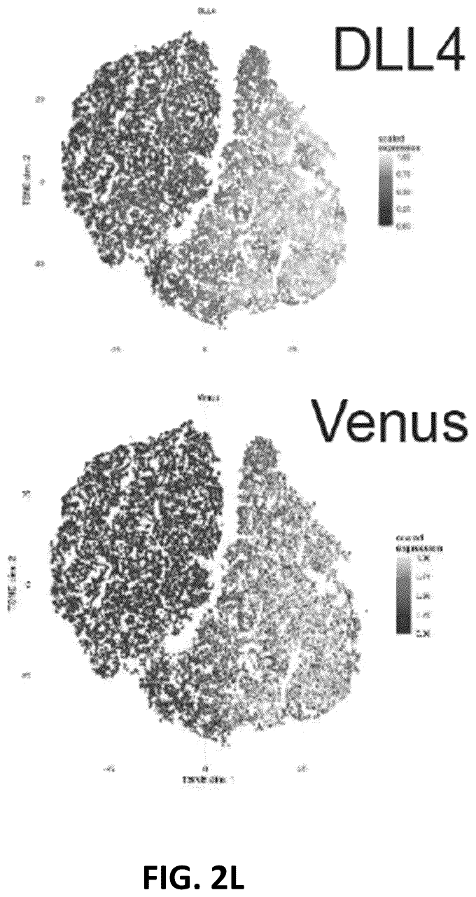

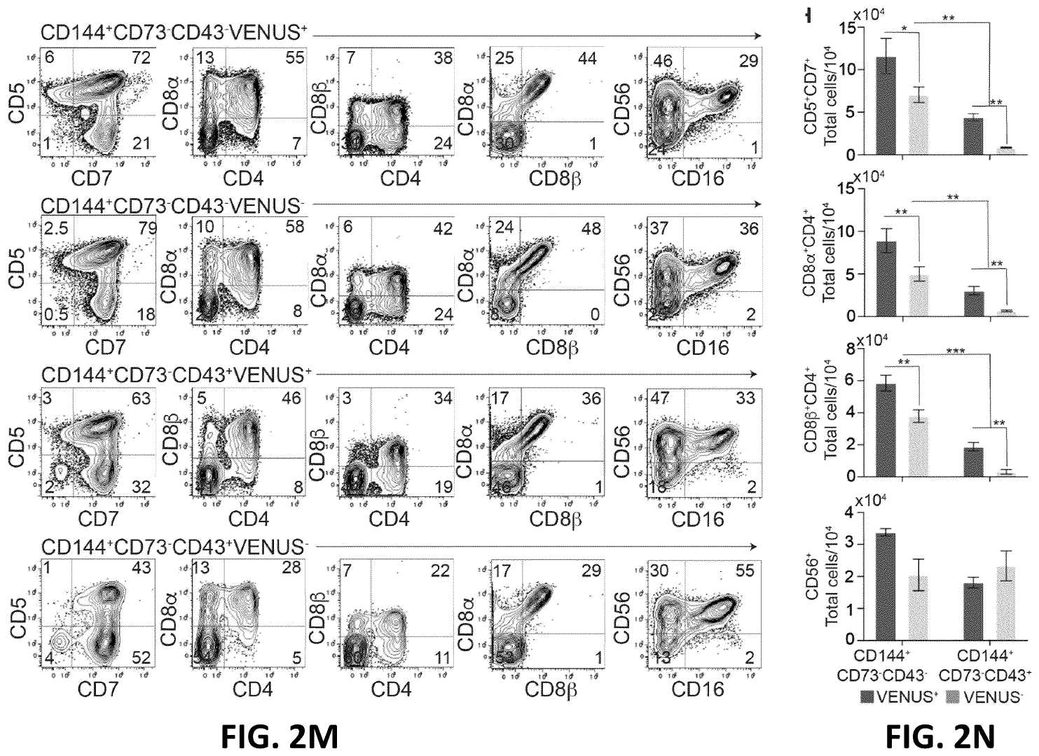

It has been postulated but not shown in the art that the delta-like canonical Notch ligand 4 (DLL4) regulates human embryonic hematopoiesis from arterial hemogenic endothelium via conserved signaling pathways such as sonic hedgehog, Wnt, and Notch (Benedito et al., 2008, BMC Dev Biol 8, 117; Boisset et al., 2010, Nature 464: 116-120; Costa et al., 2012, Trends Immunol 33: 215-223; Kim et al., 2013, Proc Natl Acad Sci USA 110: E141-150; Yamamizu et al., 2010, J Cell Biol 189: 325-338; Ivanovs et al., 2014, Stem Cell Reports 2: 449-456; Kennedy et al., 2007, Blood 109: 2679-2687). In vitro, hematopoietic differentiation of hPSCs occurs through generation of a VEC+CD43−CD73−DLL4+ arterial type HE which requires NOTCH signaling to undergo endothelial-to-hematopoietic transition (EHT) (Kumar et al., 2018, J Immunol 202: 770-776; Park et al., 2018, Cell Rep 23: 2467-2481; Uenishi et al., 2018, Nat Commun 9: 1828). Thus, DLL4 is a marker for identifying definitive hematopoietic wave from hPSC cultures (Kumar et al., 2018, J Immunol 202: 770-776; Park et al., 2018, Cell Rep 23: 2467-2481; Uenishi et al., 2018, Nat Commun 9: 1828). However, a direct progenitor-progeny link between arterial specification and definitive hematopoiesis has never been demonstrated and remains unclear. DLL4 is one of the earliest markers of the arterial lineage. A noncoding region (fragment 2) located within the third intron of DLL4 is an established arterial-specific enhancer (Sacilotto et al., 2013, Proc Natl Acad Sci USA 110: 11893-11898; Wythe et al., 2013, Dev Cell 26: 45-58). ATAC and H3K4me3 analysis has confirmed activity of DLL4 intron 3 during HE phase in response to SOX17 overexpression (FIG. 2A). Experiments were first carried out to generate a fluorescent DLL4 reporter to track real-time AHE-specific mapping of hematopoietic differentiation. The DLL4 third intron enhancer was introduced into the construct with gene-encoding VENUS fluorescent protein as shown in FIG. 2B. Enzyme digestion and Southern Blot confirmed expression of the construct in the transfected H9 hESC cell lines (FIG. 2C).

Next, differentiation of hPSC transfected with eDLL4i3:VENUS into hemogenic endothelium (HE) was tracked based on the VENUS fluorescence. FIG. 2D shows a schematic diagram of eDLL413:VENUS hPSCs differentiation using a spheroid-based differentiation protocol (FIG. 1). Expression of VENUS was highly visible in day-5 (D5) mature HE but not D4 HE (FIG. 2F). D4 and D5 cells were sorted based on expression of the indicated hemogenic endothelium cell markers (FIG. 2G and FIG. 2I) and further sorted by VENUS expression (FIG. 2H and FIG. 2J). Subset VI of D5 cells expressing CD144+CXCR4+ were prominently VENUS fluorescent (FIG. 2J). Significantly, HPs collected from VENUS+ D5 cells had higher CFC potential than VENUS− counterpart did (FIG. 2K). DLL4:13 enhancer activity closely mirror DLL4 expression as determined by flow cytometry (FIG. 2L). D5 subset cells were sorted by VENUS expression, and by expression of other markers such as CD144, CD73 and CD43, and were subjected to T potential analysis to access their capacity to differentiate into T cells by accessing T cell marker expression (CD7, CD4, CD8β, and CD16) (FIG. 2M). It was found that CD144+CD43-CD73+ VENUS+D5 hemogenic endothelial cells generated more T cells than VENUS− cells (FIG. 2N). CD144 is VE-cadherin pan-endothelial marker, CD43 is pan-hematopoietic marker, while CD73 is used to separate CD73⁻ hemogenic endothelium from CD73⁺ non-hemogenic endothelium.

These results showed that the eDLL413:VENUS construct can be used effectively to track hematopoietic differentiation of hPSCs in vitro.

### Example 2: eDLL4i3 KO Impairs Arterial Specification and Definitive Hematopoiesis

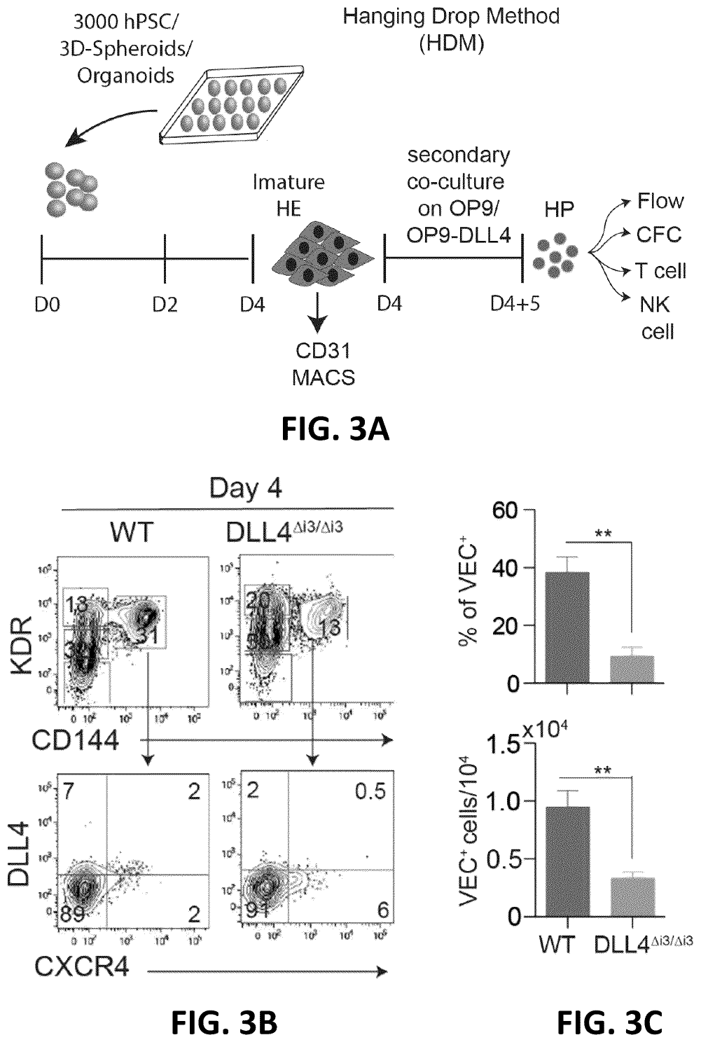

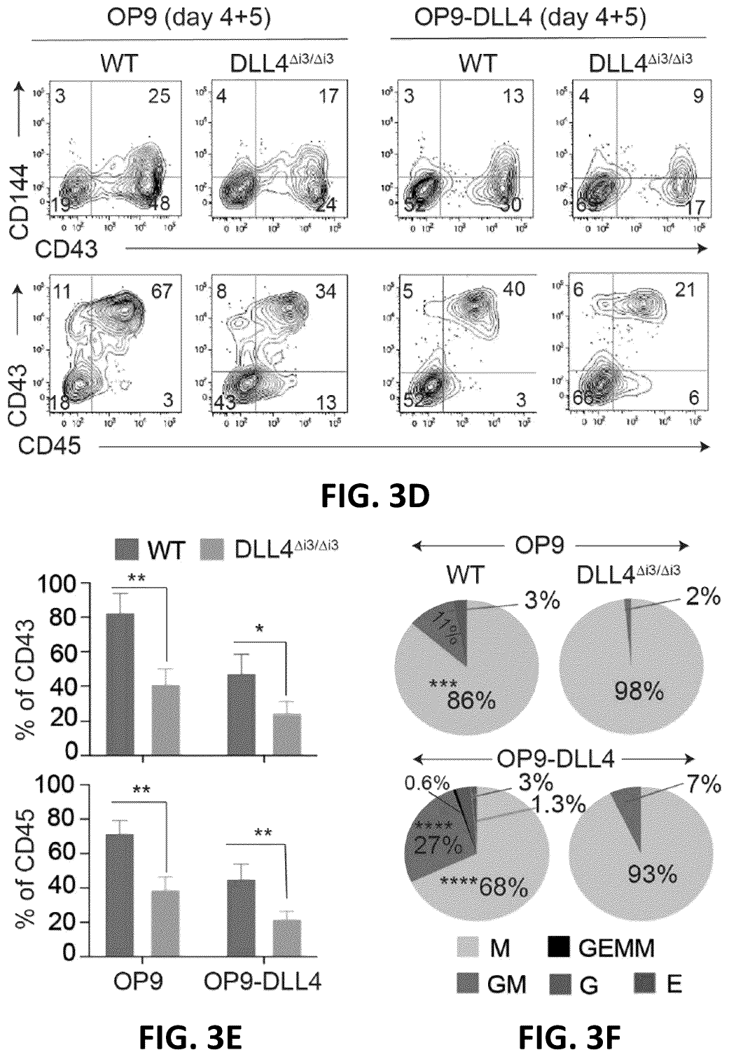

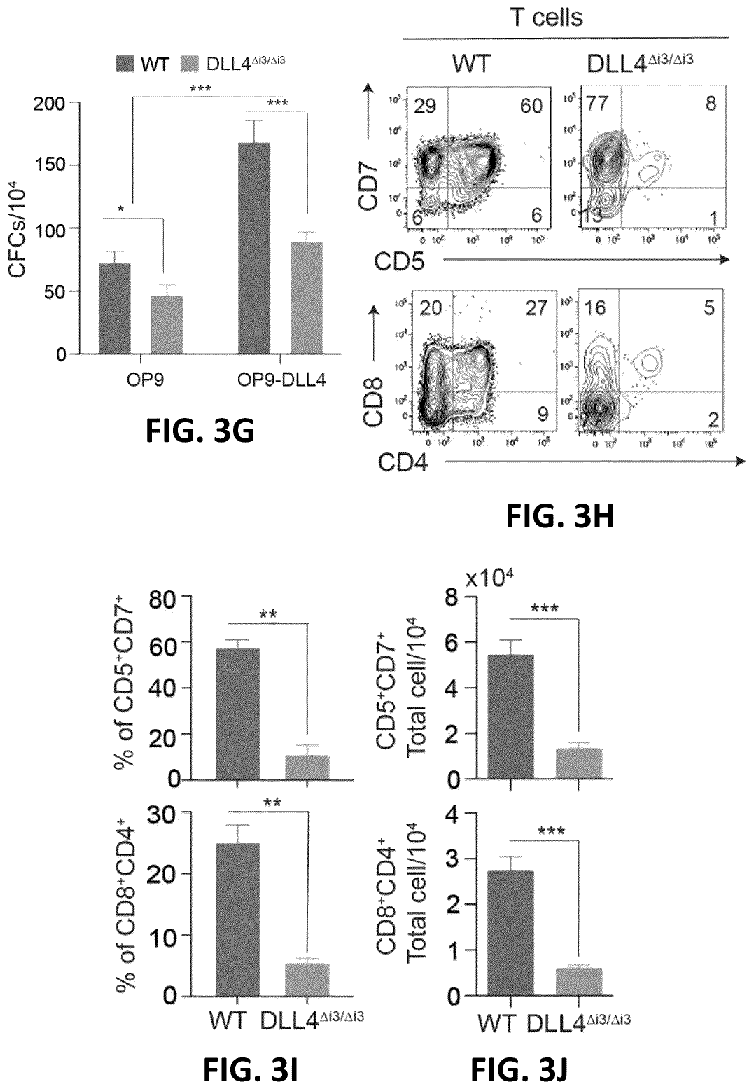

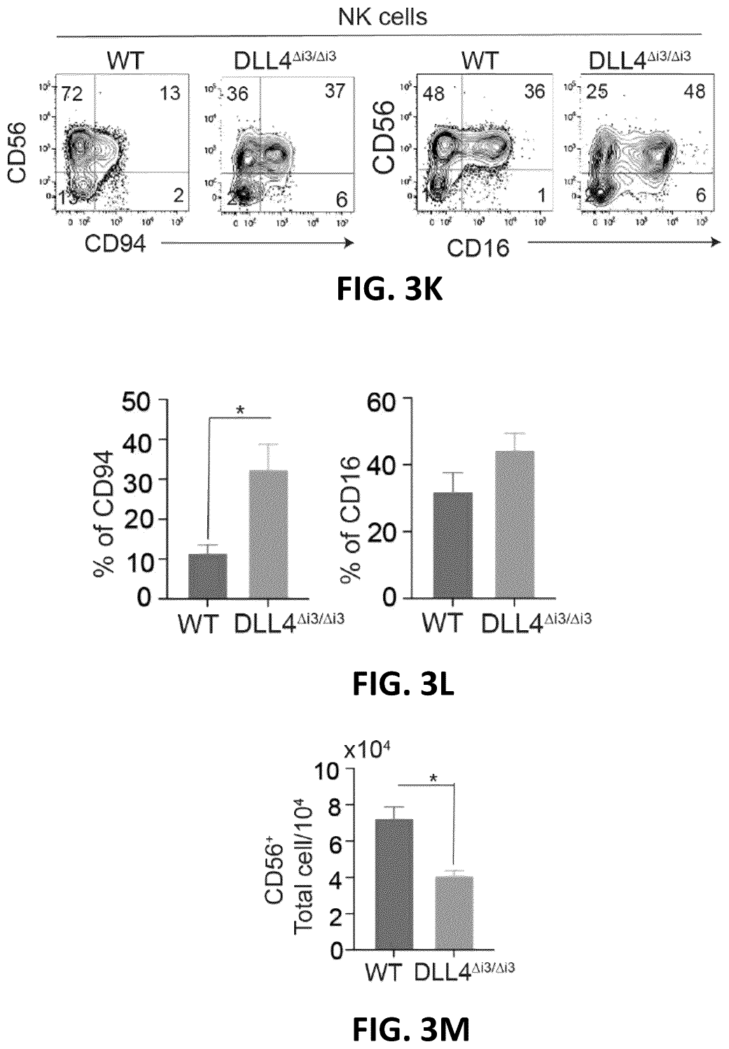

Thereafter, experiments were performed to test the role of DLL4 in regulating hematopoiesis. DLL4i3 KO cells were generated (DLL4Δi3/Δi3 or eDLL413 KO) as described above. VEC+ endothelial cells were found to be significantly lower in DLL4i3 KO compared to wild-type (WT; FIG. 3B and FIG. 3C). CFC potential from HPs collected from D4 DLL413KO cells following 5 days co-culture with OP9 and OP9-DLL4 were also blunted in eDLL413 KO (FIGS. 3D-3G). In addition, T cell potential (FIGS. 3H-3J) and NK cells potential (FIGS. 3K-3M) were diminished in cells originating from D4 eDLL413 KO HE. Similar experiments accessing arterial specification, T cell potential, and CFC potential were carried for mature HE from D5 (FIGS. 4B-4H) and D8 (FIGS. 4I-4N). In all instances, eDLL413 KO mature cells failed to achieve the same potential as WT cells.

These results showed that eDLL4i3 is an important regulator of arterial specification and T cell differentiation.

### Example 3: Transcriptionally Distinct Populations of Hemogenic Endothelial Cells and Hematopoietic Progenitors

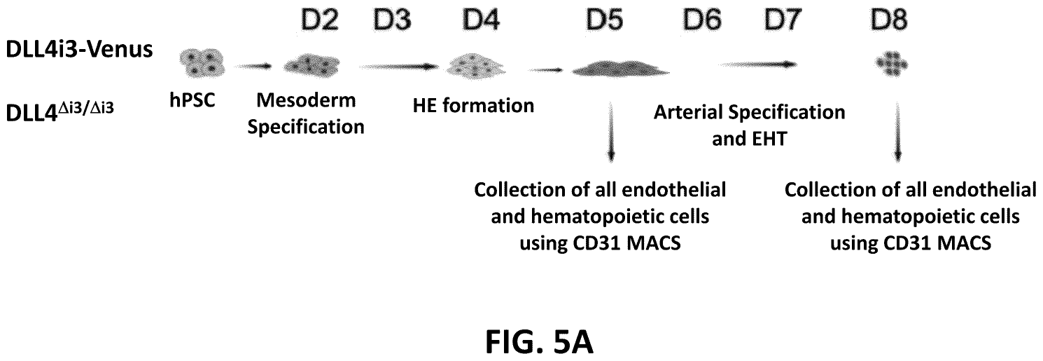

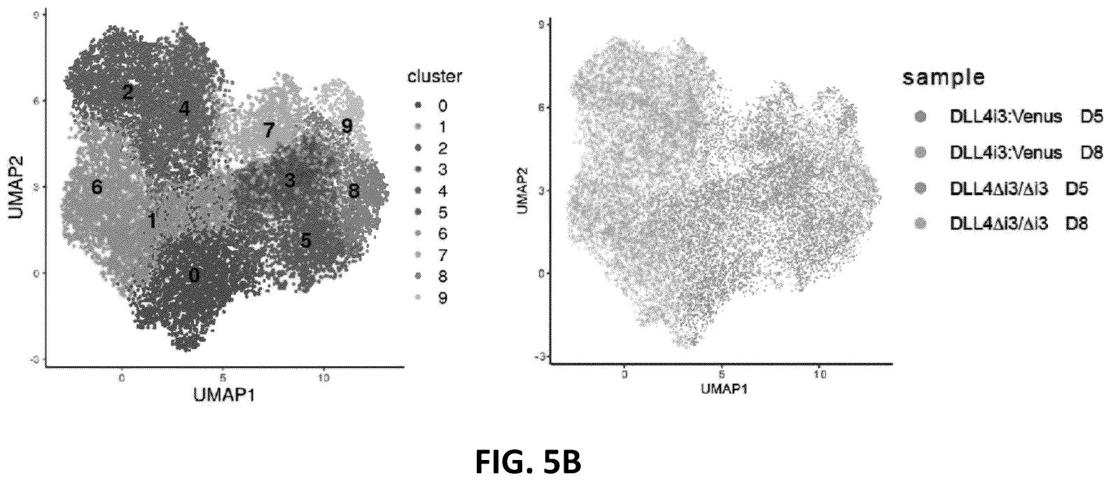

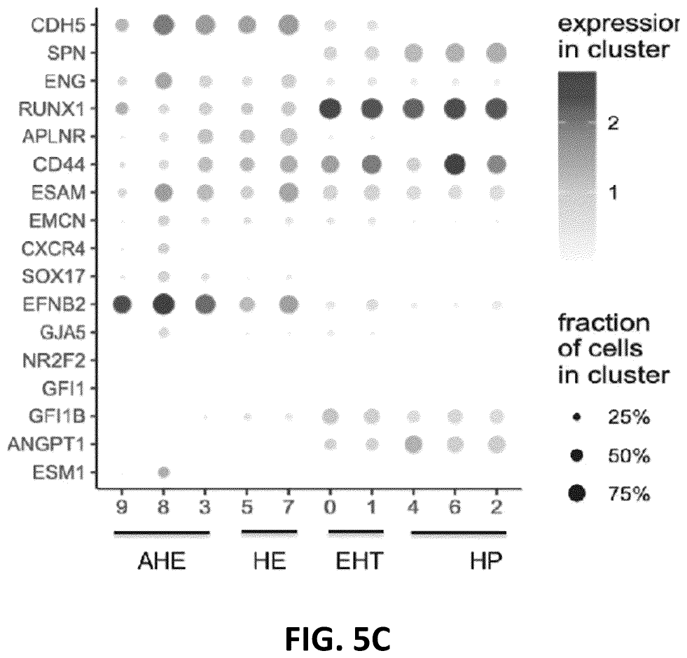

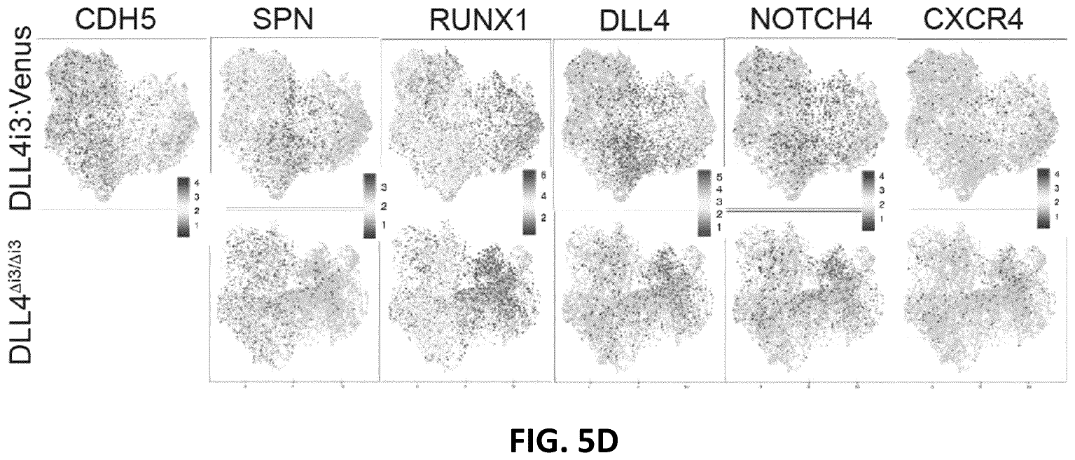

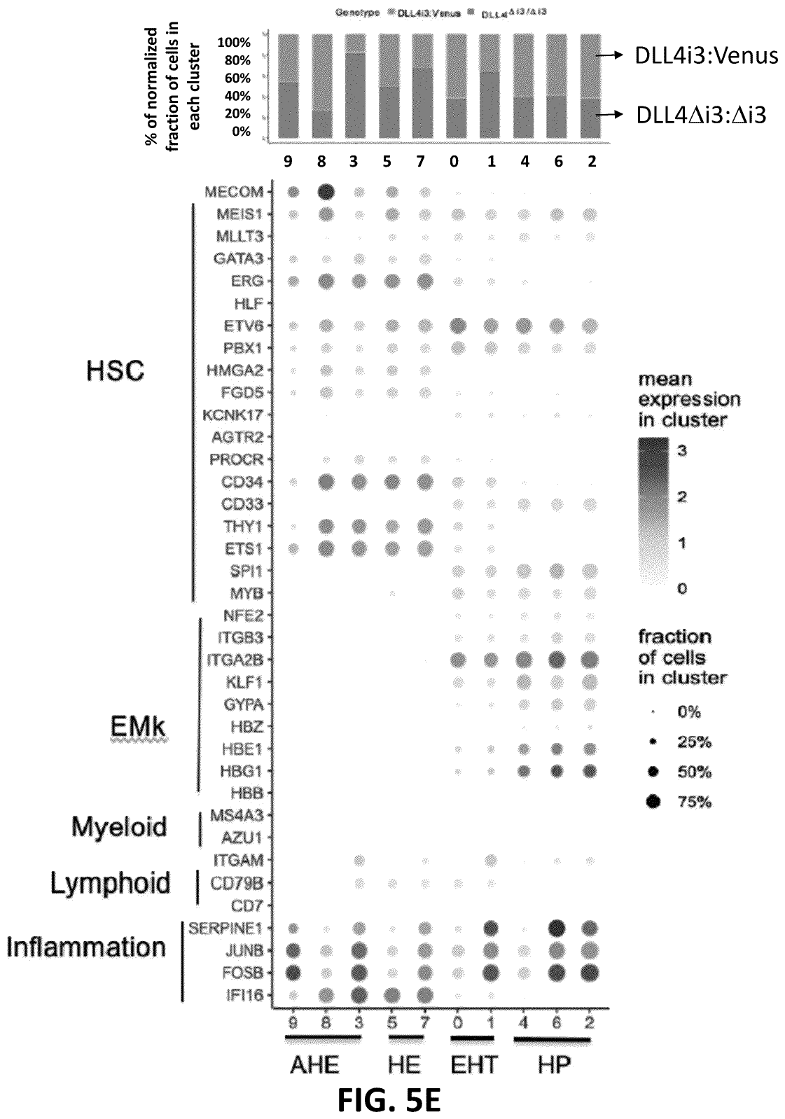

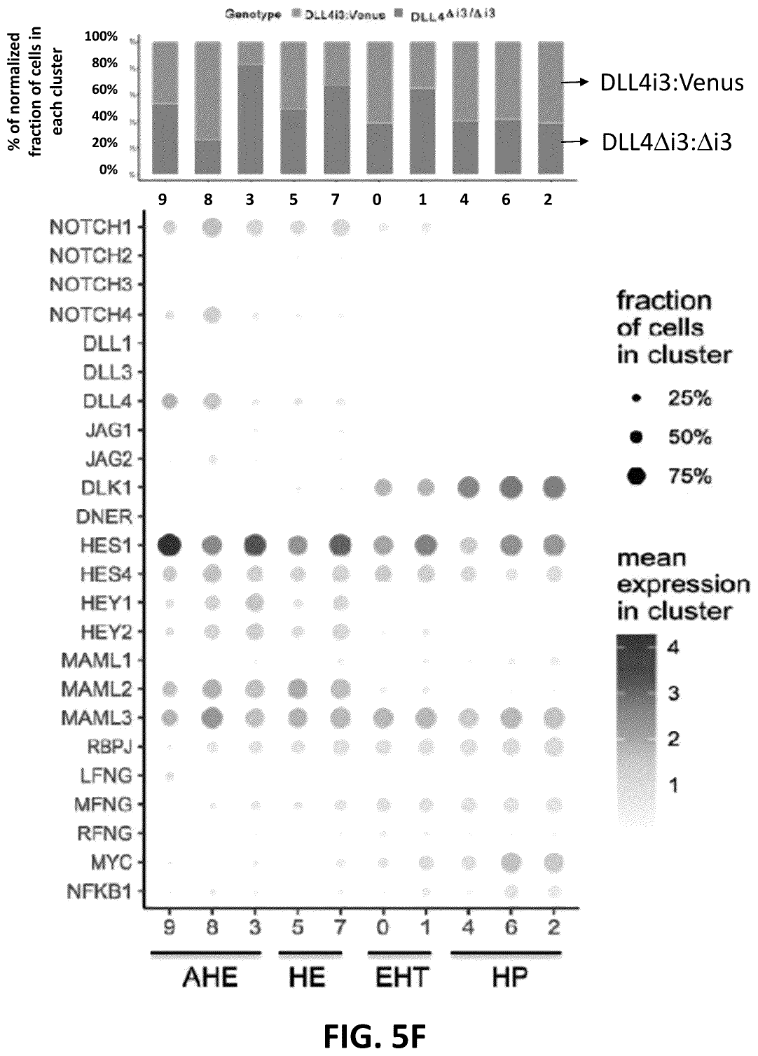

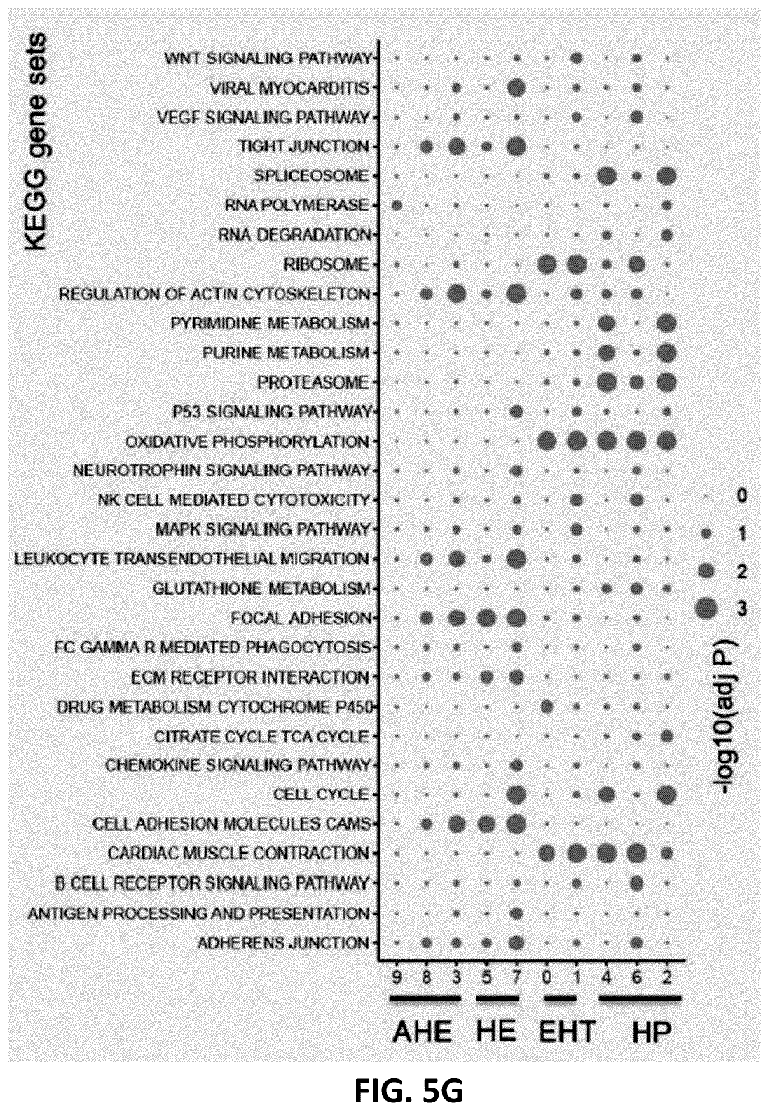

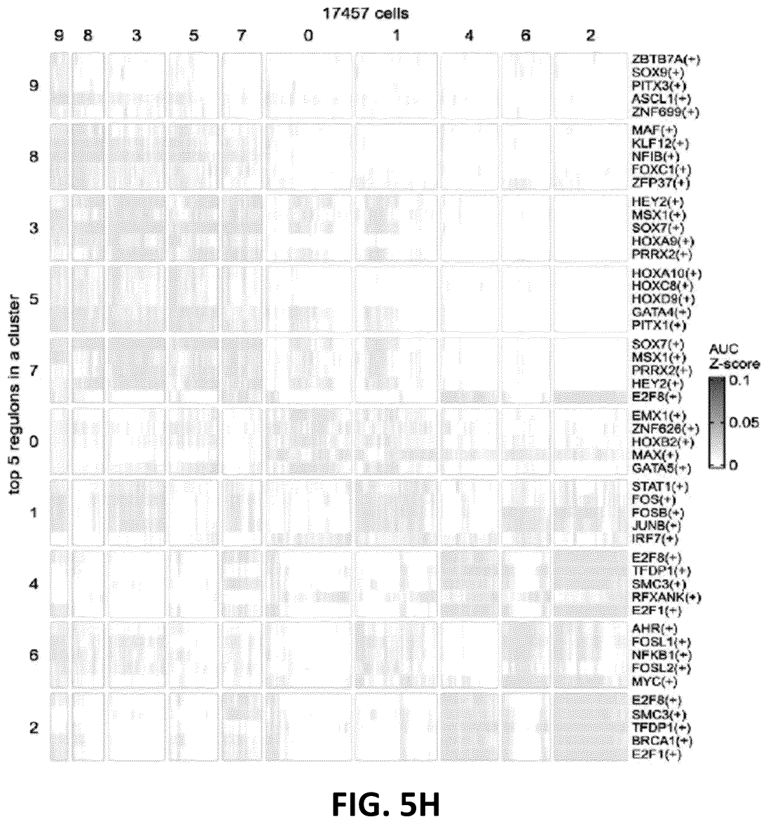

To define transcriptomic changes during EHT and early stages of hematopoietic specification, anti-CD31 antibodies were used to collect all endothelial and hematopoietic cells from D5 and D8 differentiation cultures of eDLL4i3:Venus and DLL4^{Δi3/Δi3} (or eDLL4i3 KO) hPSCs (FIG. 5A). After quality control, 17,457 (9,402 eDLL4i3:Venus and 8,055 eDLL4i3 KO) cells were analyzed. Through unsupervised clustering, 10 cell clusters (C0-C9) were distinguished and visualized using uniform manifold approximation and projection (UMAP) (FIG. 5B). Based on the expression of a panel of known pan- and arterial endothelial, and HE and hematopoietic markers, clusters 9, 8 and 3 (high EFNB2, CDH5 and RUNX1 positive and SPN negative) were identified as HE with arterial features (AHE); clusters 5 and 7 (low EFNB2, CDH5 and RUNX1 positive and SPN negative) were identified as hemogenic endothelium (HE); C0 and C1 (CDH5 and SPN positive, high RUNX1, and GFI1b) were identified as endothelial-to-hematopoietic transition (EHT); and C4, C5 and C2 (SPN positive, high RUNX1 and CDH5 negative) were identified as hematopoietic progenitors (HP) (FIG. 5C). At EHT stages, a high expression of genes associated with hematopoietic stem cells (HSC) development (MECOM, MEIS1, MLLT3, ERG, PROCR1, HMGA2, and FGD5) was observed, while upregulation of lineage-specific markers (ITGA2B, GYPA, HBG, NFE2) was observed at EHT and HP stages (FIG. 5E). Gene set enrichment analysis (GSEA) of scRNA-seq data revealed enrichment in pathways associated with cell adhesion molecules, focal adhesion and tight junction in HE and AHE cells, except C9 AHE cells which show the most enrichment in RNA polymerase pathways (FIG. 5G). Within HE cells, C7 demonstrated a selective enrichment in gene sets related to cell cycle, antigen processing and presentation, chemokine signaling, neurotrophin signaling and Fc gamma receptor mediated phagocytosis. At EHT stage (clusters 1 and 0) a marked enrichment of ribosome and oxidative phosphorylation pathways was observed, with C1 but not C0 cells showing an enrichment in NK cell mediated cytotoxicity, WNT and MAPK signaling genes. All cells at HP stage demonstrated enrichment in oxidative phosphorylation and proteasome pathways. HP cells in C2 and C4 were enriched in cell cycle, purine and pyridine metabolism, and spliceosome pathways, while C6 cells showed an enrichment in B cell receptor signaling, NK cell mediated cytotoxicity, glutathione metabolism and VEGF signaling pathways and showed no enrichment in cell cycle and purine and pyridine metabolism genes (FIG. 5G).

Similar to blood formation in the aorta-gonad mesonephros (AGM) region of human and mouse embryos, transition of AHE to HP stages was associated with dynamic changes in expression of NOTCH signaling genes. NOTCH1 and NOTCH4 receptors were highly expressed in AHE and downregulated in HE and EHT clusters (shown in FIG. 5F). Among NOTCH ligands, DLL4 expression was the most abundant and detected predominantly within AHE cluster cells. Expression of JAG2 ligand was also noted, although at much lower levels, while expression of other NOTCH ligands was negligible. However, upregulation on noncanonical NOTCH ligand DLK1 was observed following EHT. Expression of NOTCH target genes HES1 and HES4 was detected throughout all stages of development, while expression of HEY1 and HEY2 genes downregulated during transition from AHE to HP stage. NOTCH coactivators MAML2 and MAML3 were more abundant as compared to MAML1 in hemogenic endothelial populations with MAML3 expression downregulated in HP cells. Among NOTCH regulator fringe genes, the highest expression of MFNG gene was noted, which was upregulated in EHT and HP clusters.

Overall, these studies revealed a transcriptionally distinct population of hemogenic endothelial cells with arterial and non-arterial features and 3 subsets of HPs including highly proliferative C4 and C6 cells and cells with low proliferative potential enriched in genes associated with lymphoid development.

### Example 4: eDLL4i3 KO hPSCs has Higher B Cell Potential but Lower T Cell Potential than WT

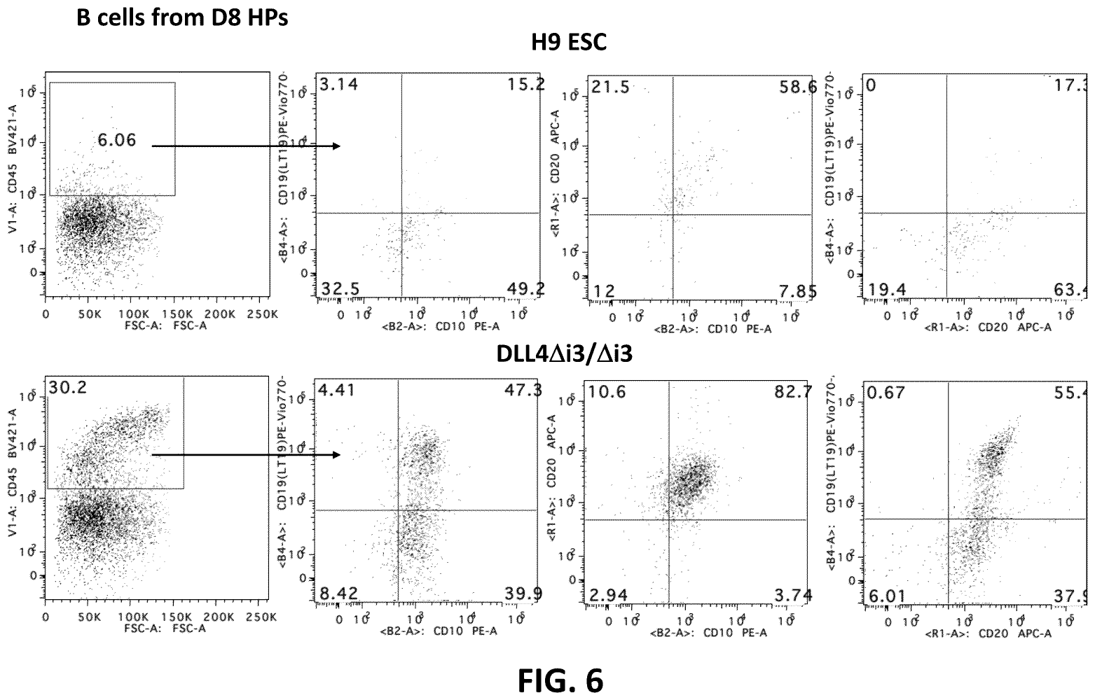

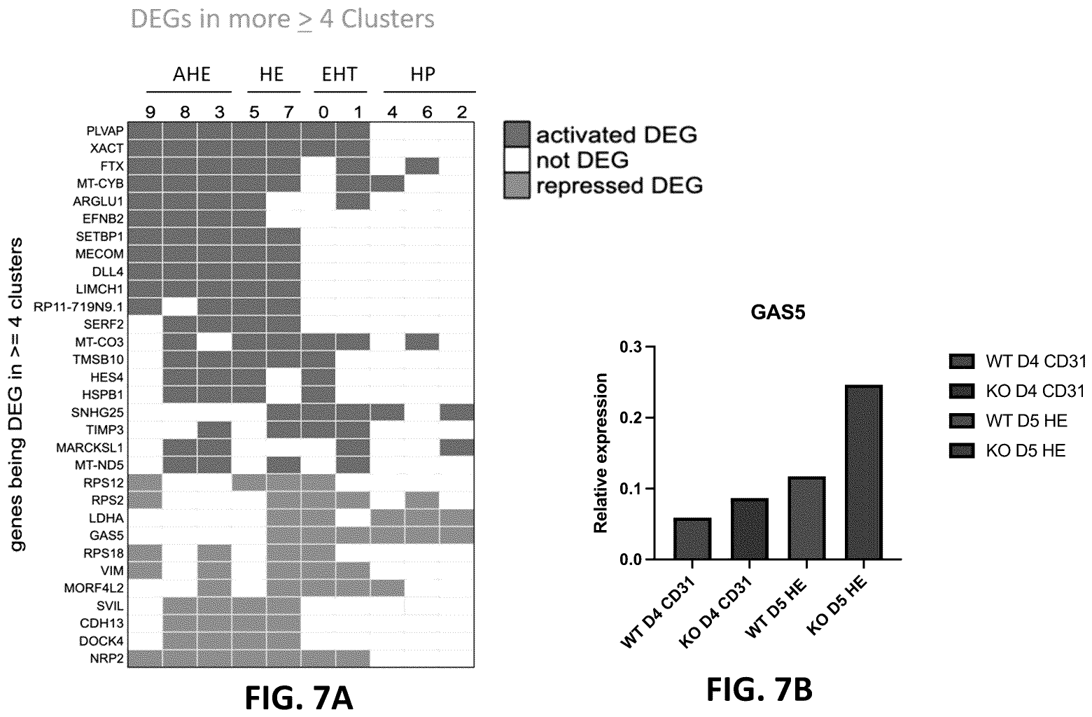

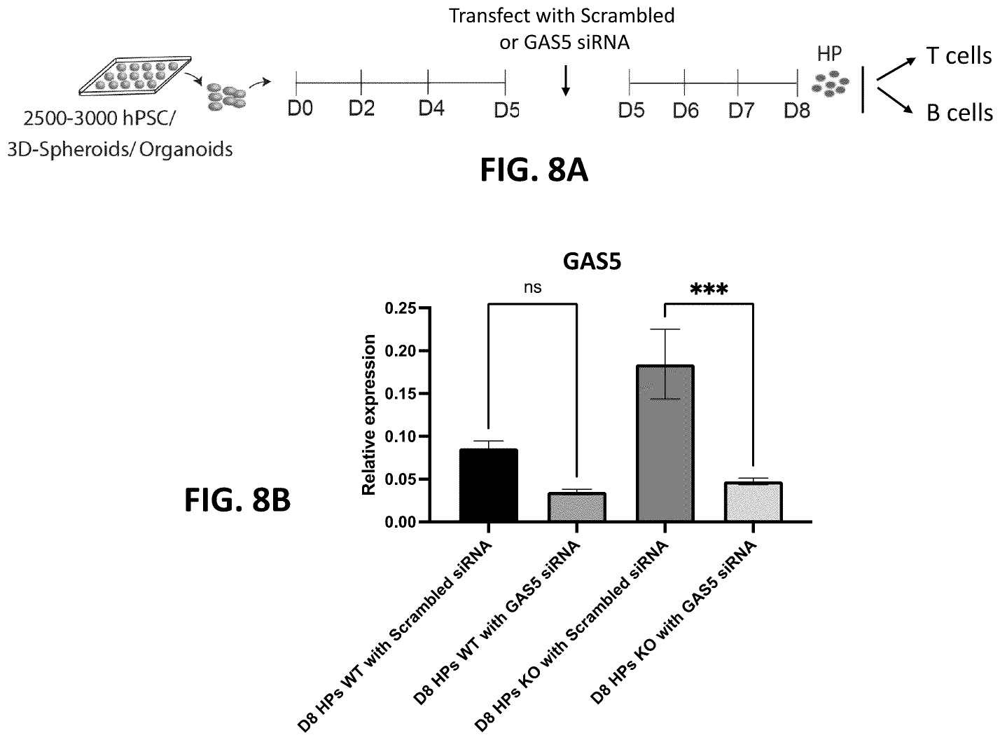

Because DLL4 regulates hematopoiesis, experiments were carried out to investigate the potential of hPSCs lacking eDLL4i3 to differentiate into immune cell types. Flow cytometric analysis of B cell markers revealed that a majority of hematopoietic progenitors (HPs) accumulated from D8 differentiation of eDLL413 KO hPSCs further expressed B cell phenotypes, which is in contrast with HPs from D8 WT (FIG. 6). RNA-seq data revealed differentially expressed genes that might contribute to this phenotype (FIG. 7A). GAS5, known to be involved in growth arrest of human peripheral blood T cells, was found to be upregulated in EHT and HP from eDLL4i3 KO as confirmed by both RNA-seq (FIG. 7A) and qPCR data (FIG. 7B). To investigate the role of GAS5 in directing immune cell differentiation, D5 mature HE was transfected with either scrambled or siRNA against GAS5 (FIG. 8A). GAS5 siRNA was sufficient to suppress upregulation of GAS5 in eDLL413 KO (FIG. 8B). Similar to previously shown FIG. 4H and FIG. 4N, T cell potential was significantly blunted in eDLL4i3 KO but strikingly, this phenomenon was reversed when GAS5 upregulation was suppressed (FIG. 9A and FIG. 9B). However, dampening GAS5 expression had no effect in reversing the B cell potential in eDLL413 KO (FIG. 10A and FIG. 10B). B cells differentiation from eDLL413 KO pluripotent stem cells was 50 times more efficient than that from wild type pluripotent stem cells (see WT-SCR vs KO-SCR in FIG. 10B). T cells differentiation from eDLL413 KO pluripotent stem cells and GAS5 expression inhibition in arterial hemogenic endothelial cells derived from the pluripotent stem cells was 5.5 times more efficient than that from wild type (WT-SCR vs KO-GAS5 in FIG. 9B), thus suggesting that GAS5 inhibition in DLL413 KO hemogenic endothelium restored their T cell potential.

### Example 5: Exogenous DLL4 Ligands do not Rescue the Lymphoid Cell Potential in DLL4KO and JAG1 Enhances T and B Cells Production Derived from DLL4KO PSC

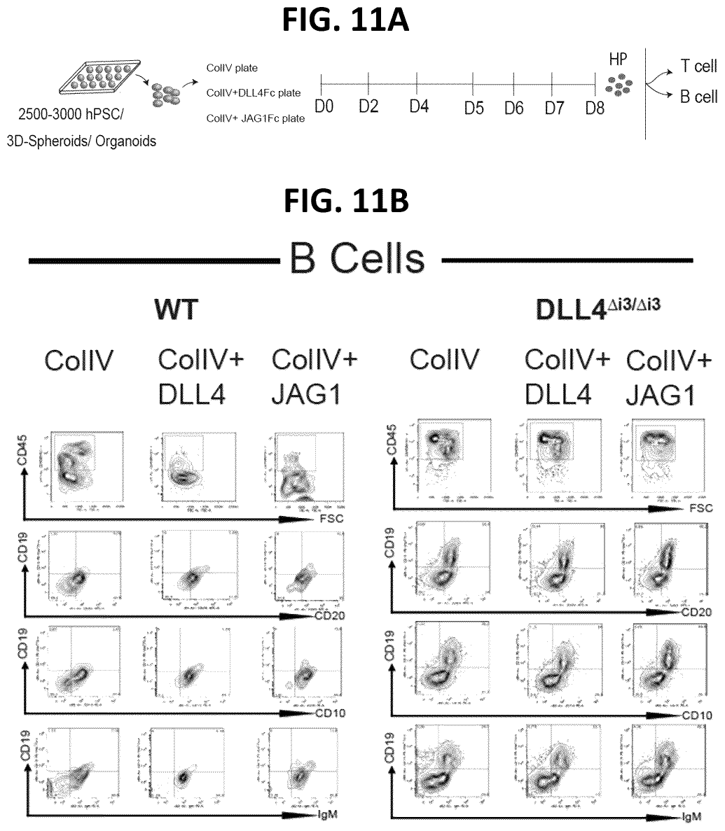

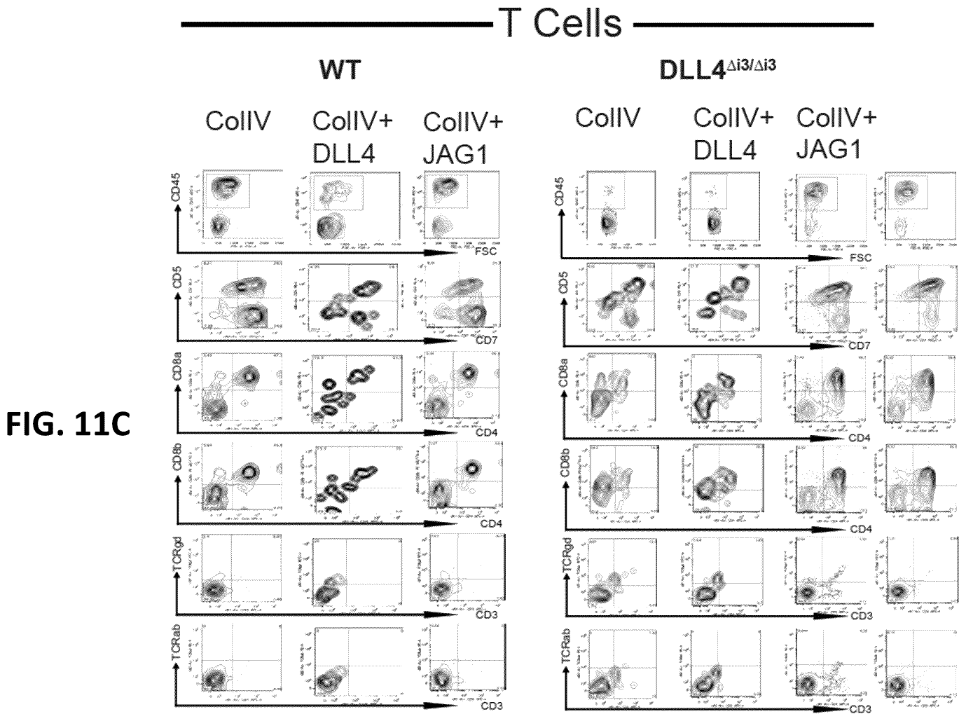

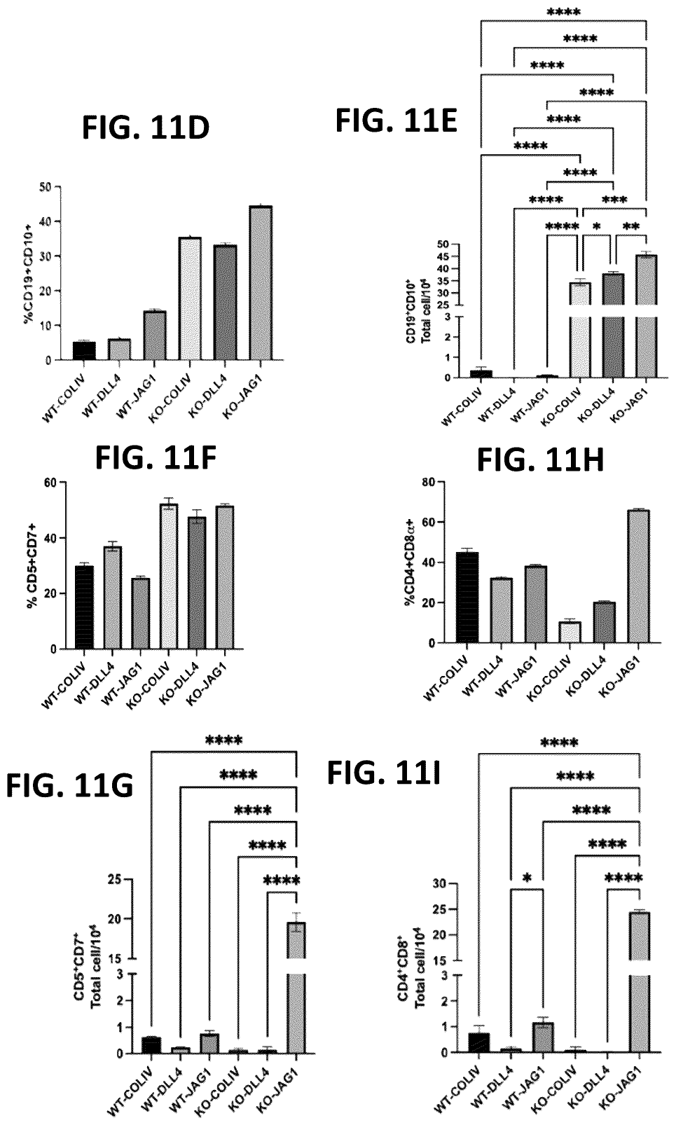

To investigate whether changes in hematopoietic potential caused by alterations in DLL4 expression following DLL413 knockout could be corrected by exposure to exogenous DLL4 ligand, differentiation was performed on plates precoated with DLL4-Fc (FIG. 11A). Culture of WT unmodified PSC on DLL4-Fc produced hematopoietic progenitors with decreased T and B cell potentials, while exposure DLL4Δi3/Δi3 to DLL4 failed to restore T cell potential but had no effect on B cell potential (FIG. 11B-11I). Because HSC development and function in aorta-gonad mesonephros (AGM) and fetal lever is highly dependent on another NOTCH ligand (JAG1; Robert-Moreno et al. EMBO J. 2008; 27(13): 1886-1895; Shao et al. Proc Natl Acad Sci U.S.A. 2023; 120(20): e2210-058120), whether T cell potential of DLL4Δi3/Δi3 can be restored in its presence was assessed. As shown in FIG. 11, exposure of WT PSCs to JAG1 slightly enhanced B cell production and had minimal effect on T cell production. However, DLL4Δi3/Δi3 PSCs in the presence of JAG1 were able to produce T cells with total output ˜ 20-fold higher as compared to WT cell controls. In addition, a slight increase was observed in B cell output from DLL4Δi3/Δi3 hematopoietic progenitors differentiated on JAG1-Fc as compared to controls. Overall, these findings indicated that PSC generation with a knockout artery-specific DLL413 enhancer can be used to enhance T and B lymphoid cell production under defined conditions in the presence of NOTCH ligand JAG1.

These results demonstrated that eDLL413 KO promoted B cell differentiation but inhibited T cell differentiation. Further dampening of GAS5 relieved inhibition of T cell potential in eDLL413 KO cells.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

1. A method of producing a spheroid containing pluripotent stem cells for hematopoietic progenitor cell differentiation, comprising the steps of:
a) dispersing pluripotent stem cells (PSCs) into a single cell suspension of spheroid medium, wherein the medium contains 25% Dulbecco's Modified Eagle medium, 25% Ham's F-12 Medium, 10% BIT 9500 supplements, 2 mM L-alanyl-L-glutamine dipeptide, 0.1 mM Non-essential Amino Acids (NEAA), 100 μM monothioglycerol (MTG), 100 μg/ml ascorbic acid, and 40% ES-Cult M3120 comprising 2.6% methylcellulose in Iscove's Modified Dulbecco's Medium,
b) apportioning the cell suspension into individual, separated droplets each comprising about 2,500-3,500 cells in 30-40 μl spheroid medium onto a solid surface in a sealable vessel, wherein the vessel is thereafter sealed and inverted wherein the droplets are hanging from the solid surface; and
c) incubating the droplet-comprising vessel for 20-24 hours in an incubator at cell-sustaining temperature and humidity conditions to produce cell-comprising spheroids.

2. A method of producing hematopoietic progenitor cells by the method of claim 1, further comprising:
a) harvesting cell-comprising spheroids with a buffer medium that is phosphate-buffered saline or iPSC culture medium by centrifugating and resuspending the spheroids in IF9S medium supplemented with bone morphogenetic protein 4 (BMP4), vascular endothelial growth factor (VEGF), fibroblast growth factor (FGF), Activin A and Rho Kinase (ROCK) inhibitor, and thereafter plating 140-160 of the spheroids on coated culture plates per 50-60 cm² surface area of the plates;
b) culturing the plated spheroids/cells under hypoxic conditions;
c) after 46-50 hours of hypoxic culture, changing the culture medium to IF9S supplemented with VEGF, FGF, BMP4, glycogen synthase kinase 3 inhibitor CHIR99021, and activin receptor-like kinase receptors inhibitor SB-431542, and again incubating the spheroids/cells under hypoxic conditions;
d) after 24 hours of hypoxic culture, changing the culture medium to IF9S medium supplemented with VEGF, FGF, and BMP4 and again incubating the spheroids/cells under hypoxic conditions;
e) after 48 hours of hypoxic culture, changing the culture medium to IF9S medium supplemented with stem cell factor (SCF), VEGF, thrombopoietin (TPO), interleukin 6 (IL6), FGF, and interleukin 3 (IL3), and incubating the spheroids/cells under normoxic conditions to facilitate endothelial to hematopoietic transition;
f) after 48 hours of normoxic culture, adding IF9S medium supplemented with SCF, VEGF, TPO, IL6, FGF, and IL3 to the culture medium and incubating the spheroids/cells under normoxic conditions to produce hematopoietic progenitors;
g) two to four days thereafter, harvesting floating hematopoietic progenitor cells from the culture medium or dislodging hematopoietic progenitor cells attached to the surface,
wherein the cells are differentiated into hemogenic endothelium cells 48 hours after step (d) and at least 50% of the hemogenic endothelium cells are arterial hemogenic endothelium cells (AHEs).

3. The method of claim 2, wherein the plates are coated with collagen, DLL4-Fc fusion protein, or JAG1-Fc fusion protein.

4. The method of claim 2, wherein VEGF is present in the culture medium at concentrations of between 25-50 ng/mL, Activin A is present in the culture medium at concentrations of between 10-15 ng/ml, ROCK inhibitor is present in the culture medium at concentrations of between 1-10 μM, CHIR99021 is present in the culture medium at concentrations of between 1-3 μM, SB-431542 is present in the culture medium at concentrations of between 3-5 μM, SCF is present in the culture medium at concentrations of between 25-50 ng/ml, TPO is present in the culture medium at concentrations of between 25-50 ng/ml, IL6 is present in the culture medium at concentrations of between 25-50 ng/ml, and IL3 is present in the culture medium at concentrations of between 5-10 ng/ml.

5. The method of claim 4, wherein in step (a), BMP4 is present in the culture medium at concentrations of between 10-50 ng/ml and fibroblast growth factor (FGF) is present in the culture medium at concentrations of between 10-50 ng/ml,
wherein in step (c) and step (d), BMP4 is present in the culture medium at concentrations of between 10-20 ng/mL and fibroblast growth factor (FGF) is present in the culture medium at concentrations of between 10-50 ng/ml, and
wherein in step (e) and step (f), fibroblast growth factor (FGF) is present in the culture medium at concentrations of between 10-20 ng/ml.

6. The method of claim 2, wherein VEGF is present in the culture medium at a concentration of 50 ng/mL, Activin A is present in the culture medium at a concentration of 15 ng/ml, ROCK inhibitor is present in the culture medium at a concentration of 1 μM, CHIR99021 is present in the culture medium at a concentration of 1 μM, SB-431542 is present in the culture medium at a concentration of 3 μM, SCF is present in the culture medium at a concentration of 50 ng/ml, TPO is present in the culture medium at a concentration of 50 ng/ml, IL6 is present in the culture medium at a concentration of 50 ng/ml, and IL3 is present in the culture medium at a concentration of 10 ng/ml.

7. The method of claim 6, wherein in step (a), BMP4 is present in the culture medium at a concentration of 50 ng/ml, and fibroblast growth factor (FGF) is present in the culture medium at a concentration of 50 ng/ml,
wherein in step (c) and step (d), BMP4 is present in the culture medium at a concentration of 10 ng/mL, and fibroblast growth factor (FGF) is present in the culture medium at a concentration of 50 ng/ml, and
wherein in step (e) and step (f), fibroblast growth factor (FGF) is present in the culture medium at a concentration of 10 ng/ml.

8. A population of hematopoietic progenitor cells (HPs) produced by the method of claim 2, wherein DLL4 expression or activity is reduced in PSC from which the HPs are produced.

9. The population of claim 8, wherein DLL4 expression is reduced in the PSCs by deletion of an artery-specific enhancer within intron 3 of DLL4.

10. The population of claim 9, wherein DLL4 expression is reduced in the PSCs by using CRISPR Cas9, ZNF, TALEN, shRNA, or siRNA.

11. The population of claim 8, wherein DLL4 activity is reduced by antibody competitively binding to Notch receptor.

12. A composition comprising the hematopoietic progenitor cells of claim 9.

13. A pharmaceutical composition comprising the composition of claim 12 and pharmaceutically or therapeutically acceptable excipients and adjuvants.

14. A method of producing T cells from the hematopoietic progenitor cells produced by the method of claim 2, further comprising:
a) culturing the hematopoietic progenitor cells with feeder cells expressing Notch-ligand delta like 4 (DLL4) in minimum essential Eagle medium with alpha modification (MEMα) supplemented 20% fetal bovine serum, SCF, FMS-like tyrosine kinase 3 ligand (FLT-3L), and interleukin 7 (IL7) for three weeks; and
b) harvesting floating cells and passaging them weekly onto the feeder cells expressing DLL4 to produce T cells.

15. The method of claim 14, wherein SCF is present in the culture medium at a concentration between 10-25 ng/ml, FLT-3L is present in the culture medium at a concentration between 5-10 ng/ml, and IL7 is present in the culture medium at a concentration between 5-10 ng/ml.

16. The method of claim 15, wherein SCF is present in the culture medium at a concentration of 10 ng/ml, FLT-3L is present in the culture medium at a concentration of 5 ng/ml, and IL7 is present in the culture medium at a concentration of 5 ng/ml.

17. The method of claim 14, further comprising reducing DLL4 expression or activity in the PSCs and reducing GAS5 expression in the HEs.

18. The method of claim 17, wherein DLL4 expression is reduced in the PSCs by deletion of artery-specific enhancer within intron 3 of DLL4.

19. The method of claim 18, wherein DLL4 expression is reduced in the PSCs by using CRISPR Cas9, transcription activator-like effector nuclease (TALEN), zinc finger nuclease (ZNF), short hairpin RNA (shRNA), or short interference RNA (siRNA).

20. The method of claim 17, wherein the DLL4 activity is reduced in the PSCs by antibody competitively binding to Notch receptor.

21. The method of claim 17, wherein GAS5 expression is reduced in the HEs by using CRISPR Cas9, TALEN, ZNF, shRNA, or siRNA.

22. The method of claim 14, further comprising reducing DLL4 expression or activity in the PSCs, and wherein the plates are coated with JAG-Fc fusion proteins.

23. The method of claim 22, wherein JAG1-Fc fusion proteins are used at a concentration between 0.5-1.5 μg per cm² of the plate's surface area.

24. A pharmaceutical composition comprising T cells produced by the method of claim 14, and pharmaceutically or therapeutically acceptable excipients and adjuvants.

25. A pharmaceutical composition comprising T cells produced by the method of claim 17, and pharmaceutically or therapeutically acceptable excipients and adjuvants.

26. A pharmaceutical composition comprising T cells produced by the method of claim 22, and pharmaceutically or therapeutically acceptable excipients and adjuvants.

27. A method of producing B cells from the hematopoietic progenitor cells produced by the method of claim 2, further comprising:
a) culturing the hematopoietic progenitor cells with feeder cells in minimum essential Eagle medium with alpha modification (MEMα) supplemented 20% fetal bovine serum, FLT3L, and interleukin 7 (IL7) for four weeks; and
b) harvesting floating cells and passaging them weekly onto the feeder cells to produce B cells.

28. The method of claim 27, wherein FLT-3L is present in the culture medium at a concentration between 5-10 ng/ml, and IL7 is present in the culture medium at a concentration between 5-10 ng/ml.

29. The method of claim 28, wherein FLT-3L is present in the culture medium at a concentration of 5 ng/ml, and IL7 is present in the culture medium at a concentration of 5 ng/ml.

30. The method of claim 27 further comprising reducing DLL4 expression or activity in the PSCs.

31. The method of claim 30, wherein DLL4 expression is reduced in the PSCs by deletion of artery-specific enhancer within intron 3 of DLL4.

32. The method of claim 31, wherein DLL4 expression is reduced in the PSCs by using CRISPR Cas9, transcription activator-like effector nuclease (TALEN), zinc finger nuclease (ZNF), short hairpin RNA (shRNA), or short interference RNA (siRNA).

33. The method of claim 30, wherein DLL4 activity is reduced in the PSCs by antibody competitively binding to Notch receptor.

34. The method of claim 27, further comprising reducing DLL4 expression or activity in the PSCs, and wherein the plates are coated with JAG-Fc fusion proteins.

35. The method of claim 34, wherein the JAG1-Fc fusion proteins are used at a concentration between 0.5-1.5 μg per cm² of the plate's surface area.

36. A pharmaceutical composition comprising B cells produced by the method of claim 27, and pharmaceutically or therapeutically acceptable excipients and adjuvants.

37. A pharmaceutical composition comprising B cells produced by the method of claim 30, and pharmaceutically or therapeutically acceptable excipients and adjuvants.

38. A pharmaceutical composition comprising B cells produced by the method of claim 34, and pharmaceutically or therapeutically acceptable excipients and adjuvants.

39. A method of producing T cells from the hematopoietic progenitor cells produced by the method of claim 2, comprising reducing DLL4 expression or activity in the PSCs and reducing GAS5 expression in the HEs, and differentiating the hematopoietic progenitor cells into the T cells thereby.

40. The method of claim 39, wherein DLL4 expression is reduced in the PSCs by deletion of an artery-specific enhancer within intron 3 of DLL4.

41. The method of claim 40, wherein DLL4 expression is reduced in the PSCs by using CRISPR Cas9, ZNF, TALEN, shRNA, or siRNA.

42. The method of claim 39, wherein DLL4 activity is reduced in the PSCs by antibody competitively binding to Notch receptor.

43. The method of claim 39, wherein GAS5 expression is reduced in the HEs by using CRISPR Cas9, ZNF, TALEN, shRNA, or siRNA.

44. A method of producing T cells from the hematopoietic progenitor cells produced by the method of claim 2, comprising reducing DLL4 expression or activity in the PSCs, and differentiating the hematopoietic progenitor cells into the T cells thereby, wherein the plates are coated with JAG-Fc fusion proteins.

45. The method of claim 44, wherein the JAG1-Fc fusion proteins are used at a concentration between 0.5-1.5 μg per cm² of the plate's surface area.

46. A pharmaceutical composition comprising T cells produced by the method of claim 39, and pharmaceutically or therapeutically acceptable excipients and adjuvants.

47. A pharmaceutical composition comprising T cells produced by the method of claim 44, and pharmaceutically or therapeutically acceptable excipients and adjuvants.

48. A method of producing B cells from the hematopoietic progenitor cells produced by the method of claim 2, comprising reducing DLL4 expression or activity in the PSCs and differentiating the hematopoietic progenitor cells into the B cells thereby.

49. The method of claim 48, wherein DLL4 expression is reduced in the PSCs by deletion of an artery-specific enhancer within intron 3 of DLL4.

50. The method of claim 49, wherein DLL4 expression is reduced in the PSCs by using CRISPR Cas9, ZNF, TALEN, shRNA, or siRNA.

51. The method of claim 48, wherein DLL4 activity is reduced by antibody competitively binding to Notch receptor.

52. A method of producing B cells from the hematopoietic progenitor cells produced by the method of claim 2, comprising reducing DLL4 expression or activity in the PSCs and differentiating the hematopoietic progenitor cells into the B cells thereby, wherein the plates are coated with JAG-Fc fusion proteins.

53. The method of claim 52, wherein the JAG1-Fc fusion proteins are used at a concentration between 0.5-1.5 μg per cm² of the plate's surface area.

54. A pharmaceutical composition comprising B cells produced by the method of claim 48, and pharmaceutically or therapeutically acceptable excipients and adjuvants.

55. A pharmaceutical composition comprising B cells produced by the method of claim 52, and pharmaceutically or therapeutically acceptable excipients and adjuvants.

56. A method of producing T cells from pluripotent stem cells (PSCs) comprising reducing DLL4 expression or activity in the PSCs and reducing GAS5 expression in hemogenic endothelium cells (HEs) derived from the PSCs, and differentiating the HEs into the T cells.

57. The method of claim 56, wherein DLL4 expression is reduced in the PSCs by deletion of an artery-specific enhancer within intron 3 of DLL4.

58. The method of claim 57, wherein DLL4 expression is reduced in the PSCs by using CRISPR Cas9, ZNF, TALEN, shRNA, or siRNA.

59. The method of claim 56, wherein DLL4 activity is reduced by antibody competitively binding to Notch receptor.

60. The method of claim 56, wherein GAS5 expression is reduced in the HEs by using CRISPR Cas9, ZNF, TALEN, shRNA, or siRNA.

61. A method of producing T cells from pluripotent stem cells (PSCs) comprising reducing DLL4 expression or activity in the PSCs and differentiating hematopoietic progenitor cells (HPs) derived from PSCs into the T cells, wherein the PSCs are cultured on JAG1-Fc fusion proteins coated plates until the HPs are obtained.

62. The method of claim 61, wherein the JAG1-Fc fusion proteins are used at a concentration between 0.5-1.5 μg per cm² of the plate's surface area.

63. A pharmaceutical composition comprising T cells produced by the method of claim 56, and pharmaceutically or therapeutically acceptable excipients and adjuvants.

64. A pharmaceutical composition comprising T cells produced by the method of claim 61, and pharmaceutically or therapeutically acceptable excipients and adjuvants.

65. A method of producing B cells from pluripotent stem cells (PSCs) comprising reducing DLL4 expression or activity in the PSCs, and differentiating the PSCs into the B cells.

66. The method of claim 65, wherein DLL4 expression is reduced in the PSCs by deletion of an artery-specific enhancer within intron 3 of DLL4.

67. The method of claim 66, wherein DLL4 expression is reduced in the PSCs by using CRISPR Cas9, ZNF, TALEN, shRNA, or siRNA.

68. The method of claim 65, wherein DLL4 activity is reduced by antibody competitively binding to Notch receptor.

69. A method of producing B cells from pluripotent stem cells (PSCs) comprising reducing DLL4 expression or activity in the PSCs and differentiating hematopoietic progenitor cells (HPs) derived from PSCs into the B cells, wherein the PSCs are cultured on JAG1-Fc fusion proteins coated plates until the HPs are obtained.

70. The method of claim 69, wherein the JAG1-Fc fusion proteins are used at a concentration between 0.5-1.5 μg per cm² of the plate's surface area.

71. A pharmaceutical composition comprising B cells produced by the method of claim 65, and pharmaceutically or therapeutically acceptable excipients and adjuvants.

72. A pharmaceutical composition comprising B cells produced by the method of claim 69, and pharmaceutically or therapeutically acceptable excipients and adjuvants.
